(19) United States
(12) Patent Application Publication
(10) Pub. No.: US 2009/0270438 A1
(43) Pub. Date: Oct. 29, 2009

(54) Novel compositions and formulations

(76) Inventors: Clive Booles, Liverpool (GB); Padriac O'Brien, Liverpool (GB); David Antony Phillip Small, Liverpool (GB); Matthew John Tyler, Liverpool (GB)

Correspondence Address: KLAUBER & JACKSON, 411 HACKENSACK AVENUE, HACKENSACK, NJ, 07601, US

(21) Appl. No.: 12/386,507

(22) Filed: Apr. 17, 2009

Related U.S. Application Data
(63) Continuation-in-part of application No. PCT/GB2007/050639, filed on Oct. 17, 2007.

(30) Foreign Application Priority Data
Oct. 18, 2006 (GB) 0620661.9
Apr. 17, 2008 (GB) 0806978.3

(51) Int. Cl. A61K 31/44

(52) U.S. Cl. 514/282

(57) ABSTRACT

There is provided according to the invention a non-pressurised pharmaceutical liquid solution spray composition comprising: (i) buprenorphine; and a solvent comprising ethanol which composition is substantially free of chloride. There is also provided according to the invention a non-pressurised pharmaceutical liquid solution spray formulation comprising: (i) buprenorphine; (ii) a solvent comprising ethanol; and (iii) one or more antioxidants each of a molar ratio of antioxidant:buprenorphine between 0.2:1 and 25:1.

### RELATED APPLICATIONS

The present application is a Continuation-In-Part of co-pending PCT Application No. PCT/GB2007/050639 filed Oct. 18, 2007, which, in turn, claims priority from GB Application No. 0620661.9 filed Oct. 18, 2006; and of GB Application No. 0806978.3 filed Apr. 17, 2008. Applicants claim the benefits of 35 USC §120 as to the said PCT application, and priority under 35 USC §119 as to the said GB applications, and the entire disclosures of all applications are incorporated herein by reference in their entireties.

### FIELD OF THE INVENTION

This invention relates to compositions and formulations of buprenorphine especially pump spray compositions and formulations suitable for transmucosal, particularly sublingual, delivery.

### BACKGROUND OF THE INVENTION

Buprenorphine, with structure shown below, is a partial agonist of opiate receptors which is widely used for the treatment of moderate to severe pain or in the treatment of opiate dependence.

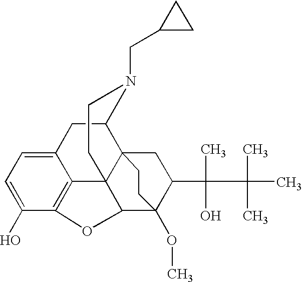

Buprenorphine is often described as a partial agonist (receptor stimulator)/antagonist (prevents receptor stimulation). It has important actions on two types of opiate receptors in the brain. Many of the most common opioid effects, such as euphoria, respiratory effects and reduced pain sensation, are caused by stimulation of the mu receptor. Buprenorphine stimulates this receptor, albeit at lower intensity than other opiates such as heroin or methadone. This lower level of stimulation is of benefit clinically in people with respiratory compromise but require opioid medication, such as the elderly.

Buprenorphine is also an antagonist of the kappa opioid receptor, which is associated with some of the negative effects experienced in withdrawal, particularly depression. As buprenorphine inhibits stimulation of this receptor it may produce feelings of well-being. Finally, its disassociation from these receptors is slow, leading to a long duration of action, allowing once daily dosing and sometimes dosing every two days, making buprenorphine a versatile treatment option in treatment of drug addiction.

A number of presentations of buprenorphine are currently available. Low-dose sub-lingual tablets, containing 0.2-0.4 mg of the drug as hydrochloride, are sold under the brand name Temgesic and are normally used for analgesic purposes. Temgesic brand of buprenorphine hydrochloride is also available as ampoules for intramuscular or slow intravenous injection. The most common formulation of buprenorphine used for the treatment of opiate dependence is sublingual tablets containing 0.4, 2 and 8 mg buprenorphine hydrochloride and available under the brand name Subutex. Using a combination of tablets, doses of up to 32 mg may be administered. These tablets are specifically intended for the treatment of problem drug use in patients who are being maintained in medically assisted treatment; in the case of patients undergoing withdrawal treatment, they are administered in a gradually reducing dose. Low-dose sublingual tablets are sometimes used for the treatment of opiate dependence, in which case multiple tablets are prescribed in order to achieve the desired dose.

A liquid formulation for sub-lingual administration is described in GB2100985 (Todd). Specifically, this document describes formulations containing buprenorphine or a non-toxic salt thereof, but especially buprenorphine hydrochloride, dissolved in 20-30% v/v ethanol in water buffered to a pH of between 4.5-5.5 with 0.05-0.2 molar concentration of a buffering agent selected from citric acid/disodium hydrogen phosphate, sodium citrate/hydrochloric acid, lactic acid/disodium hydrogen phosphate, lactic acid/sodium lactate, sodium citrate/citric acid and sodium acetate/acetic acid, the concentration of buprenorphine being between 0.8 and 10 mg/ml (i.e. around 0.08-1.0% w/v) of the composition. The Examples relate to buprenorphine hydrochloride solutions containing various different concentrations of ethanol and a variety of buffers. The compositions do not appear to be sprays as the document refers to the volume of liquid that a patient can hold sublingually for a reasonable amount of time.

It is well known that the application of carefully chosen medicaments to mucosa, for example the sublingual mucosa, offers a route of administration which is capable of resulting in very rapid transmission of medicament to the bloodstream with consequent fast onset of effect. Other mucosa to which medicaments may be administered include the nasal mucosa and buccal mucosa. A number of ways of administering compositions sublingually are known. For example, tablets or liquids may be held under the tongue prior to swallowing. Another method is spray delivery. Of these various types of sublingual administration, spray delivery is preferred as it does not involve holding the composition under the tongue for an extended period of time as, for example, with a lozenge and it reduces the amount of material which is swallowed (and may enter the blood stream in a delayed manner via the gastrointestinal tract). However it is not considered desirable to spray large volumes of liquid (eg greater than around 500 μL) to the sublingual cavity.

WO01/97780 (Ross) describes a pharmaceutical composition comprising a solution of an opioid analgesic (especially fentanyl, although buprenorphine is referred to) and a propellant, for sublingual aerosol administration. The example formulations are pressurized and therefore require complex packaging and actuation technology. Also they employ halogenated propellants which may not be environmentally friendly.

Weinberg et al (1988) Clin Pharmacol Ther 44, 335-342 discusses the adsorption of various opioids including buprenorphine (presented in an aqueous phosphate buffer at pH 6.5) when administered by pipette in liquid form to the sublingual cavity.

WO01/89476 (Pinney et al) discloses buffered compositions for transmucosal delivery. Buprenorphine is mentioned in a very long list of possible active agents and is not exemplified.

Presently there are no spray compositions containing buprenorphine which have been made available commercially.

Thus an object of the present invention is to provide a spray composition containing buprenorphine for transmucosal, particularly sublingual, administration. Further objects of the invention are to provide a spray composition containing buprenorphine for transmucosal (eg sublingual) administration with good physical and chemical properties, especially good stability and low environmental impact, and good biological properties, especially rapid onset of activity and efficacy at relatively low doses. Such a composition would mitigate many of the disadvantages of prior art compositions containing buprenorphine.

### SUMMARY OF THE INVENTION

Thus according to a first aspect of the invention there is provided a non-pressurised pharmaceutical liquid solution spray composition comprising:
(i) buprenorphine; and
(ii) a solvent comprising ethanol;
characterised in that the composition is substantially free of chloride.

According to a second aspect of the invention there is provided a non-pressurised pharmaceutical liquid solution spray formulation comprising:
(i) buprenorphine;
(ii) a solvent comprising ethanol; and
(iii) one or more antioxidants each of a molar ratio of antioxidant:buprenorphine between 0.2:1 and 25:1.

### BRIEF DESCRIPTION OF THE FIGURES

FIG. 1: Dependence of apparent pH on buprenorphine base concentration in ethanol solution

FIG. 2: Dependence of apparent pH on saccharin concentration in buprenorphine base (0.2% w/v)/ethanol solution

FIG. 3: Mean Buprenorphine plasma concentration (ng/ml)

FIG. 4: HPLC trace of comparator formulation A (Initial time point)

FIG. 5: HPLC trace of comparator formulation A (6 months)

FIG. 6: HPLC trace of comparator formulation B (Initial time point)

FIG. 7: HPLC trace of comparator formulation B (6 months)

FIG. 8: LCMS trace of comparator formulation A (Initial time point)

FIG. 9: LCMS trace of comparator formulation A (12 months)

FIG. 10: LCMS trace of comparator formulation B (Initial time point)

FIG. 11: LCMS trace of comparator formulation B (12 months)

FIG. 12: UV-vis scan 700 nm-200 nm of comparator formulation B (Initial time point)

FIG. 13: UV-vis scan 700 nm-200 nm of comparator formulation B (6 weeks)

FIG. 14: UV-vis scan 700 nm-200 nm of Example 27c (Initial time point)

FIG. 15: UV-vis scan 700 nm-200 nm of Example 27c (6 weeks)

FIG. 16: Buprenorphine stability at 40° C./75% RH (UV scan at 458 nm): Blank, Examples 27a, 27b, 27c, 28a, 28b and 28c

FIG. 17: Buprenorphine stability at 40° C./75% RH (UV scan at 458 nm): Blank, Examples 29 and 30

FIG. 18: Buprenorphine degradation at 40° C./75% RH: Blank, Examples 27a, 27b, 27c, 28a, 28b and 28c

FIG. 19: Buprenorphine degradation at 40° C./75% RH: Blank, Examples 29 and 30

### DETAILED DESCRIPTION OF THE INVENTION

The composition and formulation is non-pressurised i.e. is substantially free of any propellant. Exemplary propellants to be substantially avoided include volatile substances which develop significant vapour pressure at ambient temperature and pressure such as lower alkanes (eg propane, butane and the like) and halogenated hydrocarbons such as CFCs (P12 etc) and hydrofluorocarbons (P134a, P227 etc) as well as other propellants commonly used in aerosol presentations. Use of P11 is also preferably substantially avoided. By “substantially free” or “substantially avoided” is meant that an amount of less than 5% w/w based on weight of composition and formulation is employed, suitably less than 2% eg less than 0.1% w/w. Preferably propellants are avoided altogether.

The concentration of the buprenorphine in the composition may typically vary between 0.05 and 12% w/v, more suitably 0.1-10% w/v, eg 0.1-4% w/v or especially 2-8% w/v, for example 4-8% w/v eg 4% or 8% w/v (all figures being based on weight of buprenorphine base relative to total weight of composition).

The w/v concentration of the buprenorphine in the formulation may typically vary between 0.05 and 12% w/v, more suitably 0.1-10% w/v, eg 0.1-6% w/v, particularly 0.1-4% w/v or especially 0.1-1% w/v eg around 0.2% w/v (all figures being based on weight of buprenorphine base relative to total weight of formulation).

Suitably the molar concentration of the buprenorphine of the formulation may typically vary between 1 mM and 257 mM, more suitably 2 mM and 214 mM, eg 2-86 mM or especially 2-21 mM eg around 4.3 mM.

By “substantially free of chloride” is meant that the formulation has a substantial absence of chloride in ionised (i.e. such that Cl⁻ is formed in solution) or unionised form. The reason for the substantial absence of chloride is to avoid the precipitation of buprenorphine hydrochloride which is not highly soluble in aqueous or ethanolic solvents. Thus the amount of chloride in the composition is suitably less than 3% w/w based on weight of buprenorphine eg less than 1% w/w, eg less than 0.5% w/w for example less than 0.1% w/w, especially when the pH of the composition is less than 7.

Preferably the buprenorphine is employed as base (i.e. free base) or as citrate, particularly as base (i.e. free base).

An advantage of the first aspect of the invention, and in particular of use of buprenorphine in a formulation which is substantially free of chloride, is that relatively concentrated compositions can be prepared which allows for administration of high doses of buprenorphine without using excessively large metering volumes. For example, as will be explained below, we have successfully prepared solutions of concentration 4 and 8% w/v, whereas buprenorphine hydrochloride has not proved soluble in water or ethanol at these concentrations. These higher concentrations of buprenorphine are achieved by using a solvent containing a significant amount of ethanol.

An advantage of the second aspect of the invention, and in particular of use of buprenorphine in a formulation which contains at one or more antioxidants, is improved stability over time (especially at higher temperatures) of buprenorphine formulations comprising one or more antioxidant over formulations that do not comprise antioxidant.

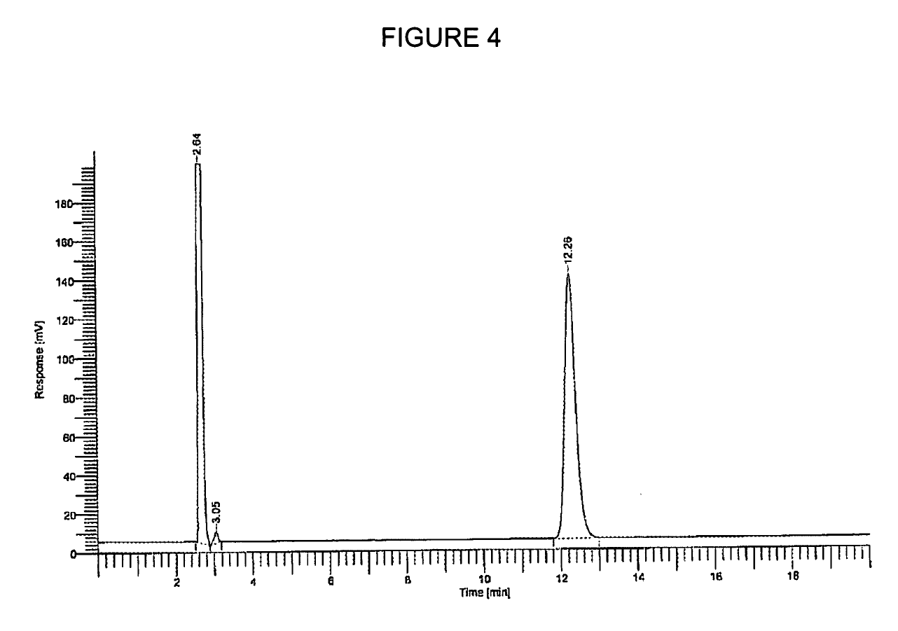

In the absence of antioxidant, the inventors have observed degradation of buprenorphine formulations, particularly ethanolic formulations accompanied by development of a yellow colour. In the absence of antioxidant, the formulations were found to be relatively stable below 4° C. However, the formulations were found to be unstable, particularly at higher temperatures (such at above 8° C.). This has obvious practical implications for storage and distribution of formulations. In the absence of antioxidant, the degradation was found to be greater at pH 5.0 than pH 8.5. (See Results and FIGS. 4-7).

The inventors have discovered that this discoloration is attributable to formation of a dimer in solution, involving the loss of oxygen, probably after a radical reaction. This route of degradation is substantially prevented, especially at higher temperatures, by incorporation of an antioxidant into the formulation. Moreover, levels of other degradation products formed over time, which may be observed by HPLC analysis are also reduced in the presence of antioxidant.

Antioxidants include, for example, alkyl gallates (other than propyl gallate), butylated hydroxyanisole (BHA), butylated hydroxytoluene (BHT), nordihydroguaiaretic acid, alpha-tocopherol, ascorbic acid and sodium metabisulfite (especially ascorbic acid). In one embodiment, the antioxidant is other than propyl gallate. In one embodiment, the antioxidant is selected from butylated hydroxyanisole (BHA), butylated hydroxytoluene (BHT), nordihydroguaiaretic acid, alpha-tocopherol, ascorbic acid and sodium metabisulfite (especially ascorbic acid).

Suitable antioxidants include butylated hydroxyanisole (BHA) butylated hydroxytoluene (BHT), alpha-tocopherol, and ascorbic acid, particularly alpha-tocopherol and ascorbic acid, particularly alpha-tocopherol and ascorbic acid, and most particularly ascorbic acid. In one particular embodiment, the antioxidant is butylated hydroxytoluene (BHT).

Ascorbic acid is commonly referred to as Vitamin C. Alpha-tocopherol is commonly referred to as Vitamin E.

Formulations of the invention will contain one or more (e.g. one or two) antioxidants. Normally one antioxidant is suitable. In one embodiment, when the formulation contains one antioxidant, said antioxidant is other than propyl gallate.

When the formulation comprises more than one antioxidant, the formulation suitably comprises two antioxidants selected from alkyl gallates, butylated hydroxyanisole (BHA), butylated hydroxytoluene (BHT), nordihydroguaiaretic acid, alpha-tocopherol, ascorbic acid and sodium metabisulfite. Most suitably the two antioxidants are alpha-tocopherol and ascorbic acid. Suitably the antioxidants are in a ratio of between 1:10 and 10:1, more suitably between 1:5 and 5:1, more suitably 1:2 and 2:1, most suitably 1:1.

Suitably the ratio of molar concentration of each antioxidant: molar concentration of buprenorphine is in the range 0.2:1 to 25:1, e.g. between 1:1 and 10:1 e.g. 2:1 and 8:1, e.g. around 4.65:1

Suitably the ratio of total molar concentration of antioxidant:buprenorphine is in the range 0.2:1 to 25:1, e.g. between 1:1 and 10:1, e.g. 2:1 and 8:1, e.g. around 4.65:1

Suitable concentrations of each antioxidant range from 0.01 mM to 250 mM, e.g. 1 mM to 100 mM, e.g. 1 mM to 50 mM e.g. 1 mM to 25 mM e.g. 5 mM to 25 mM, such as 5 mM, 10 mM and 20 mM, e.g. 10 mM and 20 mM, particularly 20 mM. In one embodiment, the concentration of each antioxidant ranges from 0.01 mM to 10 mM, such as 1 mM to 5 mM (e.g. 2.8 mM). In an alternative embodiment, the concentration of each antioxidant ranges from 50 mM to 250 mM, such as 80 mM to 230 mM (e.g. 90.8 mM or 226.9 mM).

Suitable total concentration for antioxidant(s) range from 1 mM to 100 mM, e.g. 1 mM to 50 mM e.g. 1 mM to 25 mM e.g. 5 mM to 25 mM, such as 5 mM, 10 mM and 20 mM, e.g. 10 mM and 20 mM, particularly 20 mM. In one embodiment, the concentration of total antioxidant ranges from 0.01 mM to 10 mM, such as 1 mM to 5 mM (e.g. 2.8 mM). In an alternative embodiment, the concentration of total antioxidant ranges from 50 mM to 250 mM, such as 80 mM to 230 mM (e.g. 90.8 mM or 226.9 mM).

A chelating agent such as EDTA may optionally be employed in the formulation. However, the formulation is suitably free of EDTA.

In one embodiment of the second aspect of the invention, the formulation is characterised in that the formulation is substantially free of chloride.

Some sprayable non-pressurised analgesic compositions are taught in the art, for example in WO02/094234 (Rabinowitz), WO 03/080022 (Birch) and WO 2004/071491 (Blondino). WO02/094234 relates to an opioid-containing aerosol formulation for administration by inhalation. The formulations are all aqueous solutions with no other solvent being suggested. WO 03/080022 relates to aqueous solutions comprising an analgesic for intranasal administration. The analgesic may be buprenorphine or a salt thereof but there is no teaching that the composition should not contain chloride and indeed the examples all relate to compositions comprising buprenorphine hydrochloride. There is no suggestion that ethanol could be included in the solvent. WO 2004/071491 relates to liquid aerosol formulations in which the solvent may contain ethanol. There is no suggestion that it would be advantageous to provide a chloride-free composition and all of the examples relate to formulations containing buprenorphine hydrochloride. None of the above documents suggests that buprenorphine suffers any problems with degradation in solution and specifically formation of a dimer. None of the above documents suggests that inclusion of an antioxidant would provide any specific advantage.

Generally speaking it will be desired to employ the least amount of solvent necessary (or a modest excess over that necessary) to adequately solubilise the buprenorphine such that the buprenorphine remains in solution under the conditions of likely usage or exposure.

Typically the solvent is selected from ethanol and ethanol/water mixtures. In a first embodiment of the invention ethanol is substantially the only solvent. For example the concentration of ethanol in the solvent is greater than 90% w/w eg greater than 95% w/w particularly greater than 98% w/w, for example around 100% w/w (i.e. the solvent is ethanol, the presence of any water as contaminant from the atmosphere being ignored). In this first embodiment of the invention use of water as solvent is substantially avoided, for example the water concentration is less than 10% w/w eg less than 5% w/w particularly less than 98% w/w, for example around 0% w/w (i.e. the composition and formulation is substantially free of water). As noted below, avoidance of water can be advantageous especially in formulations of buprenorphine containing citrate since we have observed that such formulations have a tendency to turn pink on storage.

In a second embodiment of both aspects of the invention the solvent comprises water as well as ethanol. For example the solvent consists of a water/ethanol mixture in which the concentration of ethanol is approximately 30-90% w/w (the balance being water) for example approximately 40-70% w/w eg around 50% w/w.

Preferably water when employed as solvent meets the USP (US Pharmacopoeia), EP (European Pharmacopoeia) “Purified Water” standards.

The pH of the solution may typically be between around 4 and 9.5 however will preferably be between around 4.5 and 9.

In a first embodiment of the first aspect of the invention the pH is between 4 and 6 eg between around 4.5 and 6 eg around 5 or between around 4 and 5 eg around 4.5. In a second embodiment of the first aspect of the invention, the pH is greater than 7 for instance between around 8 and 9.5 eg between around 8 and 9 eg around 8.5. It is envisaged that compositions at this higher pH will be more efficacious and/or have more rapid activity. Without being limited by theory it is envisaged by the inventors that buprenorphine will be more rapidly or efficiently adsorbed through the mucosa, especially the sublingual mucosa, at a pH close to the pKa of buprenorphine, which is 8.5 (Pharmaceutical Codex). Compositions of pH above 7 have not thus far been described in concrete terms, presumably due to the predominant use of buprenorphine hydrochloride and the problems of solubility of the active at higher pH. Such problems are substantially overcome by use of compositions of the invention.

In a first embodiment of the second aspect of the invention the pH is between 4.5 and 7 e.g. between around 4.5 and 7 e.g. between around 5 and 7, e.g. around 5. In a second embodiment of the second aspect of the invention, the pH is between 7 and 9 e.g. between 7 and 8.5.

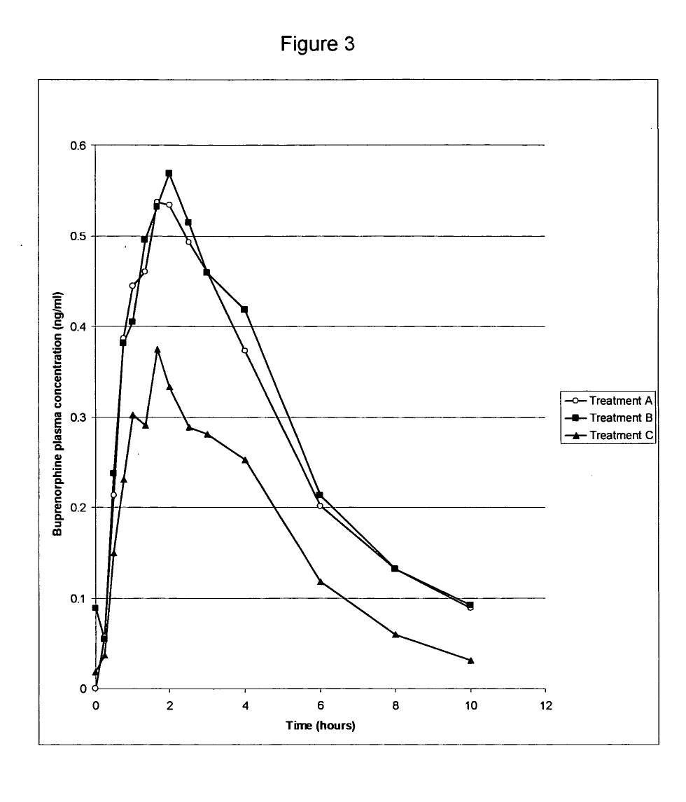

Our clinical trials have shown that the bioavailability for formulations at pH 5.0 is similar to bioavailability for formulations at pH 8.5. (See Results and FIG. 3).

We have found that in the absence of antioxidant the degradation is worse at pH 5.0 than pH 8.5. (See Results and FIGS. 4-7).

By “pH” is meant the pH reading that would be obtained using a conventional pH meter e.g. model pH 211 manufactured by Hanna Instruments Ltd and Orion 420A manufactured by Thermo Electron Corporation (i.e. in water free systems the word “pH” would be construed to mean “apparent pH”).

In order to adjust the pH buffer salts can be employed, however we have found that careful attention must be paid to the concentration of these due to the insolubility of many organic and inorganic salts in substantially ethanolic solvents. When buffers are employed, the preferred buffer system for lower pH ranges is citrate (eg sodium citrate)/citric acid which does have adequate solubility in ethanolic solvents. However citrate/citric acid is itself problematic since we have found that compositions and formulations of the invention containing citrate/citric acid and water have a tendency to turn pink on storage especially at elevated temperature. Accordingly use of buffer salts and even citrate/citric acid is preferably avoided.

Suitably the use of phosphate containing buffers (eg phosphate and protonated derivatives such as hydrogen and dihydrogen phosphate) is also avoided. Thus the amount of phosphate in the composition and formulation (eg as phosphate per se or as a protonated derivative such as hydrogen or dihydrogen phosphate) is suitably less than 3% w/w based on weight of buprenorphine eg less than 1% w/w, eg less than 0.5% w/w for example less than 0.1% w/w especially when the pH of the composition is less than 7.

We have found nevertheless that citric acid is useful to enhance the solubility of buprenorphine base in ethanolic solvents (eg to concentrations of 4% w/v or higher eg 4-8% w/w (based on total weight of composition) particularly 5-8% w/w). In such compositions the solvent may (most suitably) be essentially ethanol (eg 100% ethanol) or may (alternatively) contain water (eg ethanol/water 1:1). In order to achieve these higher concentrations typically citric acid may be employed at a concentration of around 0.1-10% w/w eg 0.2-5% w/v eg 0.2-2% w/w.

In general it should not be necessary to add buffers to formulations of the invention and therefore adding buffers is preferably avoided (ascorbic acid, sodium saccharin and saccharin are not considered to be added buffers for the purpose of this statement).

In order to address the issue of pH adjustment of buprenorphine solutions and in particular without use of conventional buffer salts or use of chloride (eg as HCl) the inventors have appreciated that it may be possible to achieve this by the use of other organic formulation components. Accordingly we undertook a careful assessment of the impact on pH on buprenorphine solutions by addition of saccharin or sodium saccharin optionally together with certain other formulation components such as menthol (eg L-menthol) or peppermint oil.

We surprisingly discovered that saccharin may be effectively employed to lower the pH of buprenorphine base compositions and formulations, and is particularly useful in achieving a pH in the range 4-6, particularly 4.5-6 eg around 5. The pH lowering effect of saccharin lessens with increased buprenorphine concentration. Addition of menthol (eg L-menthol) or peppermint oil has relatively little impact on pH when in conjunction with saccharin.

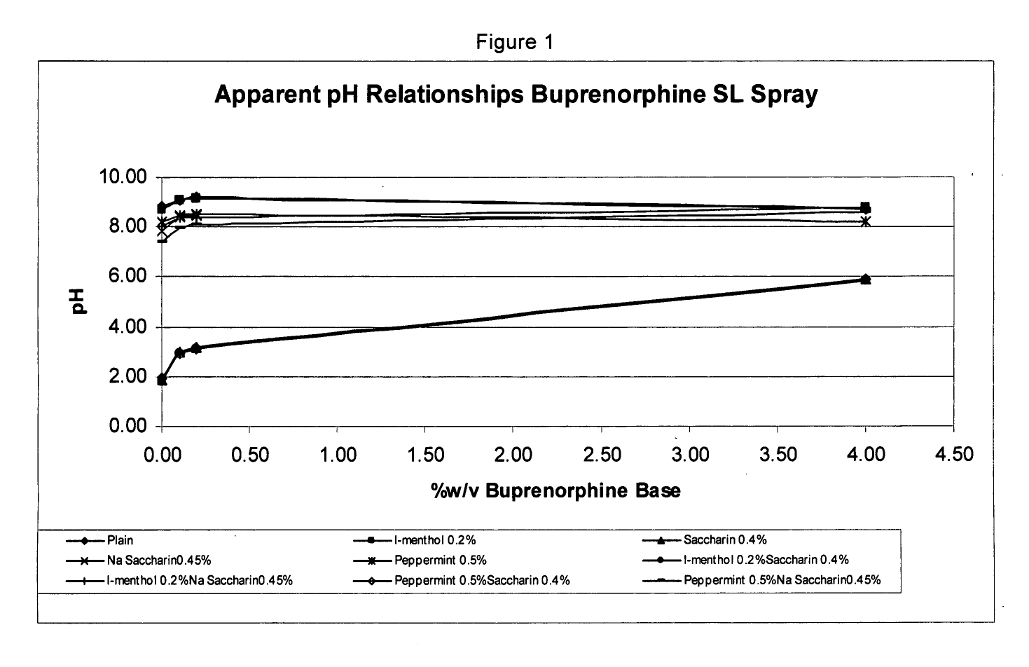

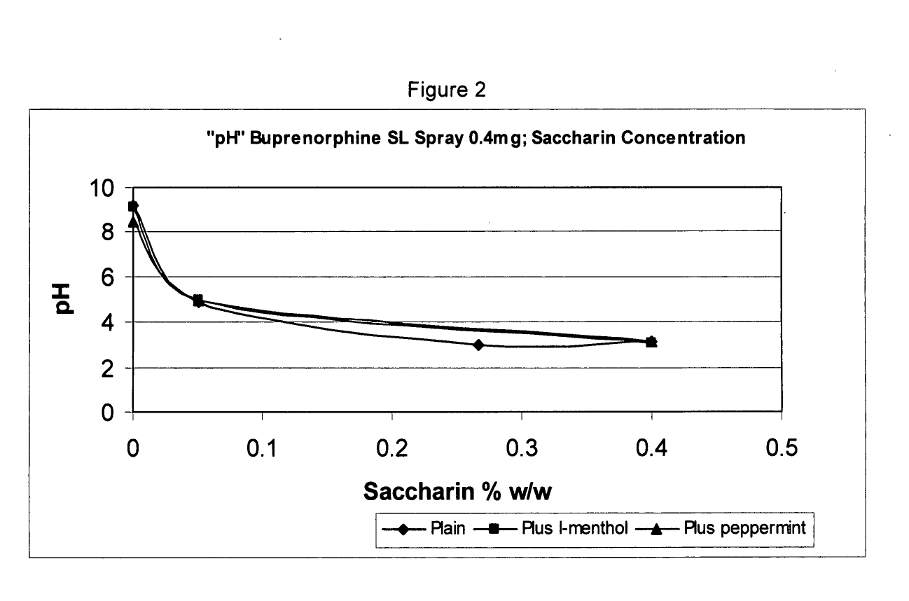

For the first aspect of the invention, we also discovered that saccharin sodium as well as menthol (eg L-menthol) and peppermint oil all have a modest but potentially useful effect on raising the pH of buprenorphine base compositions in ethanol, and is particularly useful in achieving a pH in the range between about 8 and 9.5 eg between around 8 and 9 eg around 8.5. The above mentioned results are illustrated in FIGS. 1 and 2.

For the second aspect of the invention, we also discovered that saccharin sodium as well as menthol (eg L-menthol) and peppermint oil all have a modest but potentially useful effect on raising the pH of buprenorphine base formulations in ethanol, and is particularly useful in achieving a pH in the range between about 8 and 9.5 eg between around 8 and 9 eg around 8.5 in the presence of antioxidants which are not acids (e.g. not ascorbic acid).

As well as their above mentioned useful properties in modifying the pH of the compositions and formulations, saccharin and saccharin sodium are useful as sweeteners which improve patient acceptability.

As well as their above mentioned useful properties in modifying the pH of the compositions and formulations, menthol (eg L-menthol) and/or peppermint oil are useful as flavourings and moisturing agents which may have penetration enhancing activity.

If necessary pH of the formulation can be adjusted by adding a strong acid (e.g. HCl) or strong base (e.g. NaOH).

The properties of the claimed compositions and formulations may be further improved by including therein a number of additional formulation components.

It may be desirable to include one or more of the following components in the composition and formulation
sweeteners such as saccharin, saccharin sodium, sucrose, flavouring or taste-masking agents (to improve patient acceptability),
moisturising agents (to improve patient comfort and overcome the drying tendency of ethanol and other polar organic solvents) for example peppermint oil, menthol (eg L-menthol) pineapple extract, lanolin, polypropylene glycol, polyethylene glycol.
mucoadherents (in order to increase residency time on the mucosa) for example carboxyvinyl polymers, chitosans, polyacrylic acid, gelatin, polyvinyl pyrrolidone.
preservatives (to improve long term resistance to microbial contamination) for example sodium metabisulphite, benzalkonium, Nipas.
anionic surfactants for example magnesium stearate, sodium cetostearyl sulphate, sodium lauryl sulphate, sodium oleate, sodium stearyl fumarate, sodium tetradecyl sulphate
nonionic surfactants for example glyceryl monostearate, Macrogol cetostearyl ethers, Poloxamers, polyoxyl stearates, Polysorbates, sorbitan esters, sucrose esters, Tyloxapol, propylene glycol monostearate, Quillaia, polyoxyl, caster oils, nonoxinols, lecithins and derivatives, oleic acid and derivatives, oleyl alcohol and derivatives
foaming agents for example alginic acid and salts, propylene glycol alginate, sodium lauryl sulphate, sodium cetostearyl sulphate, carbomers, hydroxyethylcellulose

In one embodiment of the first aspect of the invention, the composition may additionally include antioxidants for example alkyl gallates, butylated hydroxyanisole, butylated hydroxytoluene, nordihydroguaiaretic acid, tocopherols, Ascorbic acid, sodium metabisulphite.

Amongst the above mentioned possible additional components, it may be remarked that a preservative should not normally be necessary in view of the ethanol content of the compositions and formulations.

In accordance with best pharmaceutical principles, additional components will be avoided if not necessary.

We have observed that compositions according to the first aspect of the invention which are of higher strength (e.g. 4% w/v or above), especially those containing saccharin, have a tendency to yellow on storage, especially at higher temperatures. Accordingly a stabiliser selected from anti-oxidants (e.g. ascorbic acid/ascorbate) and/or a chelating agent (e.g. EDTA/sodium edetate) may suitably be employed.

Some of the components proposed above may already be included in the composition and formulation of the present invention for other purposes. Suitable moisturising agents include, for example, the polar organic solvents such as glycols, especially propylene glycol, and the liquid polyethylene glycols, glycerol, methylcellulose, hypromellose, hydroxypropylcellulose, and many other substituted celluloses.

As mentioned above, a versatile component, which improves the acceptability and other properties of the composition and formulation, is menthol especially L-menthol. Menthol (e.g. L-menthol), as well as flavouring the composition and formulation, has moisturising effect. It may also have effect as a penetration enhancer. Preferably menthol (e.g. L-menthol) is employed in a concentration range of 0.1% to 0.75% w/w eg around 0.2% w/w.

Peppermint oil is an alternative component which may be used in place of menthol. Peppermint is known to have incompatibilities with certain actives (eg fentanyl) however it appears to be compatible with buprenorphine. Suitably peppermint oil is employed in a concentration range of 0.1% to 0.75% w/w eg around 0.5% w/w.

In preferred embodiments of the invention, the composition and formulation contains a sweetener. In one embodiment of the invention, the sweetener is saccharin sodium. Suitably the concentration of saccharin sodium is around 0.1-0.9% w/w eg around 0.45% w/w.

In another embodiment of the invention, the composition and formulation contains saccharin. Suitably the concentration of saccharin is around 0.025-0.75% w/w, for example around 0.05-0.4% w/w eg around 0.05-0.1% w/w. As pointed out above the concentration of saccharin may be varied depending on the eventual pH desired (see FIG. 2).

A number of compositions and formulations of the invention are considered to be especially suitable.

A suitable example composition of the first aspect of the invention comprises (or consists essentially of (eg consists of)):
buprenorphine as base;
a solvent selected from ethanol and ethanol/water mixtures;

wherein the composition is substantially free of chloride; and

wherein the pH of the composition is greater than 7.

The pH of the composition may, for instance, be between around 8 and 9.5 eg between around 8 and 9 eg around 8.5.

The solvent may suitably be ethanol.

Optionally (and advantageously) such compositions comprise saccharin sodium.

Optionally (and advantageously) such compositions comprise a flavouring agent selected from menthol (e.g. L-menthol), peppermint oil and mixtures thereof.

Optionally such compositions comprise a chelating agent (eg EDTA or sodium edetate).

Optionally such compositions comprise an anti-oxidant.

Optionally hydroxide (e.g. NaOH, KOH) may be used to raise the pH if needed.

Suitably the concentration of buprenorphine base is 0.1-4% w/v.

Another suitable example composition of the first aspect of the invention comprises (or consist essentially of (e.g. consist of)):
buprenorphine as base;
a solvent selected from ethanol and ethanol/water mixtures;
saccharin;

wherein the composition is substantially free of chloride; and wherein the pH of the composition is between around 4 and 6 e.g. between around 4.5 and 6 e.g. around 5.

The solvent may suitably be ethanol.

Optionally (and advantageously) such compositions comprise a flavouring agent selected from menthol (eg L-menthol), peppermint oil and mixtures thereof.

Optionally such compositions comprise a chelating agent (eg EDTA or sodium edetate).

Optionally such compositions comprise an anti-oxidant.

Suitably the concentration of buprenorphine base is 0.1-4% w/v.

Another suitable example composition of the first aspect of the invention comprises (or consist essentially of (eg consist of)):
buprenorphine as base at a concentration of 4% w/v or more;
a solvent selected from ethanol and ethanol/water mixtures;
citric acid;

wherein the composition is substantially free of chloride; and wherein the pH of the composition is between around 4 and 6 e.g. between around 4 and 5 eg around 4.5.

The solvent may suitably be ethanol.

Optionally (and advantageously) such compositions comprise a flavouring agent selected from menthol (e.g. L-menthol), peppermint oil and mixtures thereof.

Optionally (and advantageously) such compositions comprise saccharin.

Optionally such compositions comprise a chelating agent (eg EDTA or sodium edetate).

Optionally such compositions comprise an anti-oxidant.

Suitably the concentration of buprenorphine base is 4-8% w/v.

A suitable example formulation of the second aspect of the invention comprises (or consists essentially of (e.g. consists of)):
buprenorphine in the form of its base;
a solvent selected from ethanol and ethanol/water mixtures;
one or more antioxidants each of a molar ratio of antioxidant:buprenorphine between 0.2:1 and 25:1 and

wherein the pH of the formulation is between 7 and 9.

The pH of the formulation may, for instance, be between around 7 and 8.5 eg around 8 or around 8.5.

Suitably none of the antioxidants is an acid (e.g. ascorbic acid).

The solvent may suitably be ethanol.

Optionally (and advantageously) such formulations are substantially free of chloride.

Optionally (and advantageously) such formulations comprise saccharin sodium.

Optionally (and advantageously) such formulations comprise a flavouring agent selected from menthol (e.g. L-menthol), peppermint oil and mixtures thereof.

Optionally such formulations comprise a chelating agent (e.g. EDTA or sodium edetate).

Optionally hydroxide (e.g. NaOH, KOH) may be used to raise the pH if needed.

Suitably the concentration of buprenorphine base is 0.1-4% w/v e.g. 0.1-1% w/v.

Another suitable example formulation of the second aspect of the invention comprises (or consists essentially of (e.g. consists of)):
buprenorphine in the form of its base;
a solvent selected from ethanol and ethanol/water mixtures;
one or more antioxidants, one or more of which is an acid (e.g. ascorbic acid), each of a molar ratio of antioxidant:buprenorphine between

0.2:1 and 25:1 and

wherein the pH of the formulation is between around 4.5 and 7 e.g. between around 4.5 and 7 e.g. between around 5 and 7, e.g. around 5.

The solvent may suitably be ethanol.

Optionally (and advantageously) such formulations are substantially free of chloride.

Optionally (and advantageously) such formulations comprise saccharin sodium.

Optionally (and advantageously) such formulations comprise a flavouring agent selected from menthol (e.g. L-menthol), peppermint oil and mixtures thereof.

Optionally such formulations comprise a chelating agent (e.g. EDTA or sodium edetate).

Optionally hydroxide (e.g. NaOH, KOH) may be used to raise the pH if needed.

Suitably the concentration of buprenorphine base is 0.1-4% w/v e.g. 0.1-1% w/v.

Another suitable example formulation of the second aspect of the invention comprises (or consists essentially of (e.g. consists of)):
buprenorphine in the form of its base;
a solvent selected from ethanol and ethanol/water mixtures;
one or more antioxidants, each of a molar ratio of antioxidant:buprenorphine between 0.2:1 and 25:1;
saccharin;

wherein the pH of the formulation is between around 4.5 and 7 e.g. between around 4.5 and 7 e.g. between around 5 and 7, e.g. around 5.

The solvent may suitably be ethanol.

Optionally (and advantageously) such formulations are substantially free of chloride.

Optionally (and advantageously) such formulations comprise a flavouring agent selected from menthol (e.g. L-menthol), peppermint oil and mixtures thereof.

Optionally such formulations comprise a chelating agent (eg EDTA or sodium edetate).

Suitably the concentration of buprenorphine base is 0.1-4% w/v e.g. 0.1-1% w/v.

A process for preparation of compositions of the first aspect of the invention comprises:

(a) taking buprenorphine as base and a solvent comprising ethanol optionally containing the other formulation ingredients (eg saccharin, saccharin sodium, menthol, peppermint oil etc) and dissolving the buprenorphine in the solvent; or

(b) taking buprenorphine as base and a solvent comprising ethanol and dissolving the buprenorphine in the solvent, then adding the other formulation ingredients (eg saccharin, saccharin sodium, menthol, peppermint oil, etc); or

(c) the process of (a) or (b) in which the pH of the solvent is adjusted (e.g. with citric acid) once all the other formulation ingredients are mixed together.

A process for preparation of formulations of the second aspect of the invention comprises:

(a) taking buprenorphine in the form of its base and antioxidant and a solvent comprising ethanol optionally containing the other formulation ingredients (eg saccharin, saccharin sodium, menthol, peppermint oil, etc.) and dissolving the buprenorphine and antioxidant in the solvent; or

(b) taking buprenorphine in the form of its base and antioxidant and a solvent comprising ethanol and dissolving the buprenorphine and antioxidant in the solvent, then adding the other formulation ingredients (e.g. saccharin, saccharin sodium, menthol, peppermint oil, etc.); or

(c) taking buprenorphine in the form of its base and a solvent comprising ethanol containing antioxidant and optionally containing the other formulation ingredients (e.g. saccharin, saccharin sodium, menthol, peppermint oil, etc.) and dissolving the buprenorphine in the solvent; or

(d) taking buprenorphine in the form of its base and a solvent comprising ethanol and dissolving the buprenorphine in the solvent, then adding the other formulation ingredients (antioxidant and eg saccharin, saccharin sodium, menthol, peppermint oil, etc.); or

(e) the process of (a), (b), (c) or (d) in which the pH of the solvent is adjusted once all the other formulation ingredients are mixed together.

Process (b) is preferred.

Amongst the advantages of the claimed compositions and formulations is the fact that by being non-pressurised they avoid the issues associated with using propellant, such as their manufacturing disadvantages and their potential environmental impact (many propellants are “greenhouse gases”). The solution compositions and formulations of the invention are homogenous and have limited or no susceptibility to dose-to-dose variation. Furthermore compositions and formulations of the present invention are characterised by good long-term physical and chemical stability.

The compositions and formulations of the invention are preferably administered transmucosally (particularly sublingually) as a spray. The compositions and formulations are expected to be well tolerated when administered to the sensitive sublingual mucosa and the sublingual spray administration will result in rapid onset of the therapeutic effect of the buprenorphine.

Thus according to a third aspect of the invention there is provided a metered dose dispensing system comprising a sealed container containing a composition of the first aspect of the invention or a formulation of the second aspect of the invention fitted with a metering pump, an actuator and a channeling device. The metered dose dispensing system is preferably adapted for transmucosal (particularly sublingual) administration.

Although in principle the container for the pharmaceutical liquid composition or formulation may contain a single dose of buprenorphine (which may, nevertheless be a divided dose), preferably the container will contain a plurality of doses (e.g. 20 to 200 doses) of buprenorphine.

Although the composition or formulation could be packaged in a suitable pharmaceutical grade, plastics container, such a container would be relatively easy to open for abuse of the product. Therefore a glass container would be more suitable. Glass would shatter if attempts were made to open the pack, rendering the contents either lost or unusable due to glass fragments. Preferably the glass container will be coated on the exterior with a suitable moulded film of plastic to protect against shattering. For example the film may be of polypropylene. The material may be coloured and contain a UV absorber. The container glass may be colourless or more suitably may be provided with a UV protective colouring, for example amber colouring. Optionally, the interior of the container can be coated to enhance stability of the product. Coatings include polymers and lacquers but also silicone dioxide as an unreactive coating can be used to line the inside of the container.

Since the composition and formulation is non-pressurised, it is suitably administered to the patient by pump action. Thus the metering dose dispensing system suitably contains a metering pump permitting a metered dose of the composition or formulation to be administered as a spray.

Suitable metering pumps include those adapted for dispensation with the container in the upright or inverted orientation. Preferably the metering chamber is adapted for dispensation with the container in the upright orientation since this facilitates administration under the tongue. Accordingly the metering chamber will be in communication with the composition or formulation by means of a dip-tube.

The metering pump is suitably a non-venting type. Suitable materials of construction include polypropylene and polyethylene. Example metering pumps are those manufactured by Valois (eg VP3, VP6, VP7 and VP7D) and for example those illustrated in International Patent Application No. WO01/66089. Other conventional pumps include those from Rexam (eg SP270) and Calmar (eg Accupump or Mistette Mk II).

Preferably the actuator will be designed to deliver a transmucosally (particularly a sublingually) effective dose. The pump may suitably be manually actuated, although assisted actuation using stored energy (e.g. spring or gas) may be contemplated.

For a secure seal, the pump is suitably crimped onto the container neck. Suitable sealing materials e.g. thermo plastic crimp gaskets suitable for the purpose will be employed. In addition, a suitable aluminium ferrule purposely designed for crimping on to glass containers may suitably be employed. Suitable grade stainless steel springs will preferably be adopted.

The metering pump will administer a metered volume of formulation. Suitable metering volumes are 10-1000 μL, more suitably 50-250 μL, e.g. 100 μL or 200 μL, particularly 200 μL.

A channeling device is provided to direct the liquid sprayed from the metered dose dispensing area to the appropriate part of the mouth e.g. to the sublingual cavity or if desired to the nose. Channeling devices are suitably fabricated from moulded plastics. A number of channeling devices adapted to administer sprays to the mouth or nose are known to persons skilled in the art e.g.

| | | | |
|---|---|---|---|
| | Nasal | | |
| Spray Buttons | Actuators | Throat Actuators | Dental Actuators |

Valois

| | | | |
|---|---|---|---|
| 155 GPC | CB 18 NAC | 132 C GP | 251 EB 406 |
| 155 GPCS | CB 18 NAL | 132 L GP | 251 EB 407 |
| 165 GPC | CB 18 NEC | 139 foldable | |
| 165 GPCS | CB 18 W NAC | 137 C | |
| 852 | CB 19 | 137 L | |
| A3 | 147 NE | 251 EB 408 | |

Calmar

| | | | |
|---|---|---|---|
| Standard Head | 2-piece Nasal | Short Throat | Capillary Tube |
| | 3-piece Nasal | Medium Throat | |
| | | Long Throat | |
| | | Articulated Throat | |

Rexam

| | | | |
|---|---|---|---|
| 9590 | 4345 | 9180 | 9410 |
| | AA5733 | 4234 | 5200 |
| | AB3960 | AA9994 | |
| | 5561 | 5860 | |
| | 4095 | 585 | |
| | AA8238 | 4325 | |

Compositions and formulations of the invention are useful in treatment or prevention of opiate dependency and abuse, particularly in the treatment or prevention of dependency on opiates such as heroin and for analgesic purposes eg for the treatment of moderate to severe pain. Thus in a further aspect of the invention there is provided a method of treatment or prevention of opiate dependency and abuse or pain which comprises administering to a subject in need thereof an effective amount of a composition or formulation of the invention.

In order to lessen the risk of abuse with the product, suitably the container or the dispensing system may be provided with features to prevent tampering. In particular, the container or the dispensing system may suitably be provided with features to prevent or discourage access to the reservoir and/or to prevent administration of more than one dose of buprenorphine at one time.

The dispensing system, in particular the actuator, may, for example, be provided with a lock-out feature to prevent administration of a second dose within a specified time interval of the first. Lock-out features are, for example, described in US2006191532, WO03097141 and WO0232487.

Typically a patient is treated by administration transmucosally (eg sublingually) of 1 to 4 actuations eg 1 or 2 actuations from the spray pump. Another advantage of mucosal spray delivery is the ability to easily titrate patients by 1 or 2 doses as required by a single actuation. This is not the case with other forms of drug delivery (patches, lozenges, tablets, suppositories).

Pharmaceutical compositions and formulations of the invention are useful in the treatment of animals, particularly non-human mammals (for example domestic or livestock animals) as well as humans. Accordingly pharmaceutical uses, for example uses in the treatment of pain, may be extended to veterinary uses. Dosages and methods of administration (e.g. the spray actuator design) will be adapted for the intended recipient as would be known to a skilled person.

### EXAMPLES RELATING TO THE FIRST ASPECT OF THE INVENTION

### Examples 1-4

Compositions were prepared as follows:

| | Ex 1 | Ex 2 | Ex 3 | Ex 4 |
|---|---|---|---|---|
| Buprenorphine base (% w/v) | 0.2 | 0.1 | 0.1 | 0.1 |
| Levomenthol (% w/w) | 0.2 | 0.2 | — | — |
| Peppermint oil (% w/w) | — | — | — | 0.5 |
| Sodium saccharin (% w/w) | 0.45 | — | 0.45 | — |
| Ethanol anhydrous (%) | to 100 | to 100 | to 100 | to 100 |
| Measured pH | 8.38 | 9.08 | 8.36 | 8.43 |

The compositions formed a clear colouriess solution at 4° C. and remained so after 1 month storage at these temperatures.

### Examples 5-8

Compositions were prepared as follows:

| | Ex 5 | Ex 6 | Ex 7 | Ex 8 |
|---|---|---|---|---|
| Buprenorphine base (% w/v) | 0.2 | 0.2 | 0.2 | 0.1 |
| Levomenthol (% w/w) | — | 0.2 | — | — |
| Peppermint oil (% w/w) | — | — | 0.5 | 0.5 |
| Saccharin (% w/w) | 0.05 | 0.05 | 0.05 | 0.40 |
| Ethanol anhydrous (%) | to 100 | to 100 | to 100 | to 100 |
| Measured pH | 4.92 | 4.93 | 4.97 | 2.94 |

The compositions formed a clear colourless solution at 4° C. and remained so after 2 weeks storage (Example 8) or 1 month storage (Examples 5-7) at these temperatures.

### Examples 9-12

Compositions were prepared as follows:

| | Ex 9 | Ex 10 | Ex 11 | Ex 12 |
|---|---|---|---|---|
| Buprenorphine base (% w/v) | 0.1 | 0.1 | 0.2 | 0.1 |
| Levomenthol (% w/w) | 0.2 | — | — | — |
| Peppermint oil (% w/w) | — | 0.5 | 0.5 | — |
| Sodium saccharin (% w/w) | 0.45 | 0.45 | 0.45 | — |
| Ethanol anhydrous (%) | to 100 | to 100 | to 100 | to 100 |
| Measured pH | 8.36 | 7.98 | 8.08 | 9.04 |

The compositions formed a clear colourless solution at 4° C. and remained so after 1 month storage at these temperatures.

### Examples 13-16

Compositions were prepared as follows:

| | Ex 13 | Ex 14 | Ex 15 | Ex 16 |
|---|---|---|---|---|
| Buprenorphine base (% w/v) | 4 | 4 | 4 | 4 |
| Levomenthol (% w/w) | 0.2 | — | — | 0.2 |
| Peppermint oil (% w/w) | — | 0.5 | — | — |
| Sodium saccharin (% w/w) | — | — | — | — |
| Saccharin (% w/w) | — | — | — | — |
| Ethanol anhydrous (%) | to 100 | to 100 | to 100 | to 100 |
| Measured pH | 8.63 | 8.35 | 8.67 | NM |

NM = not measured

Example 13: the composition formed a clear colourless solution at 4° C. and remained so after 2 months storage at these temperatures.

Example 14: the composition formed a clear colourless solution at 4° C. and a clear very light yellow solution at 40° C. after 2 months storage at these temperatures.

Examples 15, 16: the compositions formed a clear colourless solution at 4° C. and a clear light yellow solution at 40° C. after 3.5 months storage at these temperatures.

### Examples 17-20

Compositions were prepared as follows:

| | Ex 17 | Ex 18 | Ex 19 | Ex 20 |
|---|---|---|---|---|
| Buprenorphine base (% w/v) | 4 | 4 | 4 | 4 |
| Levomenthol (% w/w) | — | 0.2 | — | 0.2 |
| Peppermint oil (% w/w) | — | — | 0.5 | — |
| Sodium saccharin (% w/w) | — | — | — | 0.45 |
| Saccharin (% w/w) | 0.40 | 0.40 | 0.40 | |
| Ethanol anhydrous (%) | to 100 | to 100 | to 100 | to 100 |
| Measured pH | 5.89 | 5.85 | 5.87 | 8.77 |

NM=not measured

Examples 17 and 18: the compositions formed a clear colourless solution at 4° C. and a dark yellow solution at 40° C. after 3.5 months storage at these temperatures.

Example 19: the composition formed a clear light yellow solution at 4° C., a clear yellow solution at 25° C. and a clear dark yellow solution at 40° C. after 3.5 months storage at these temperatures.

Example 20: the composition formed a clear colourless solution at 4° C., a clear light yellow solution at 25° C. and a clear yellow solution at 40° C. after 2 months storage at these temperatures.

### Examples 21-24

Compositions were prepared as follows:

| | Ex 21 | Ex 22 | Ex 23 | Ex 24 |
|---|---|---|---|---|
| Buprenorphine base (% w/v) | 8 | 8 | 8 | 4 |
| Levomenthol (% w/w) | — | — | — | — |
| Peppermint oil (% w/w) | — | — | — | — |
| Sodium saccharin (% w/w) | — | — | — | — |
| Saccharin (% w/w) | — | — | — | — |
| Citric acid (% w/w) | 4.13 | 8.22 | 4.11 | 2.05 |
| Water/ethanol 1:1 w/w (% | | | to 100 | to 100 |
| w/w) | | | | |
| Ethanol anhydrous (% w/w) | to 100 | to 100 | — | — |
| Measured pH | 4.83 | 4.20 | 4.55 | 4.64 |

Examples 21 and 22: the compositions formed a clear colourless solution at 4, a light yellow solution at 25° C. and a yellow solution at 40° C. after 3 months storage at these temperatures.

Example 23: the composition formed a clear colourless solution at 4° C. and a clear pink solution at 40° C. after 3 months storage at these temperatures.

Example 24: the composition formed a clear colourless solution at 4° C. and a clear light pink solution at 40° C. after 3 months storage at these temperatures.

### Example 25

The dependence of apparent pH on buprenorphine base concentration in ethanol solution was investigated for various compositions. The results are shown in FIG. 1.

The most striking observation is that saccharin has a significant effect on the composition apparent pH, which decreases with buprenorphine base concentration. The overlapping profiles at markedly lower pH was obtained from the 3 compositions containing (i) saccharin; (ii) saccharin with peppermint oil; and (iii) saccharin with L-menthol.

### Example 26

The dependence of apparent pH on saccharin concentration in buprenorphine base (0.2% w/v)/ethanol solution was investigated for various compositions. The results are shown in FIG. 2.

### EXAMPLES RELATING TO THE SECOND ASPECT OF THE INVENTION

### Comparator Formulations

| | |
|---|---|
| Material | % w/w |

Comparator Formulation A (pH 8.5)

| | |
|---|---|
| Buprenorphine | 0.253 |
| Levomenthol | 0.200 |
| Saccharin | 0.450 |
| Sodium | |
| 100% Ethanol | 99.097 |

Comparator Formulation B (pH 5.0)

| | |
|---|---|
| Buprenorphine | 0.253 |
| Levomenthol | 0.200 |
| Saccharin | 0.050 |
| 100% Ethanol | 99.497 |

The following example formulations were prepared for comparison with Comparator Formulation.

### Example Formulation 27 (Ascorbic acid, pH 5.0)

| a) | | b) | | c) | |
|---|---|---|---|---|---|
| Material | % w/w | Material | % w/w | Material | % w/w |
| Bupre- | 0.253 | Bupre- | 0.253 | Buprenorphine | 0.253 |
| norphine | | norphine | | | |
| Levomenthol | 0.200 | Levomenthol | 0.200 | Levomenthol | 0.200 |
| Saccharin | 0.050 | Saccharin | 0.050 | Saccharin | 0.050 |
| 100% | 99.410 | 100% | 99.320 | 100% Ethanol | 99.140 |
| Ethanol | | Ethanol | | | |
| Ascorbic | 0.087 | Ascorbic | 0.177 | Ascorbic acid | 0.357 |
| acid | | acid | | (20 mM) | |
| (5 mM) | | (10 mM) | | | |

### Example Formulation 28 (Alpha-tocopherol, pH 5.0)

| a) | | b) | | c) | |
|---|---|---|---|---|---|
| Material | % w/w | Material | % w/w | Material | % w/w |
| Bupre- | 0.253 | Bupre- | 0.253 | Buprenorphine | 0.253 |
| norphine | | norphine | | | |
| Levomenthol | 0.200 | Levomenthol | 0.200 | Levomenthol | 0.200 |
| Saccharin | 0.050 | Saccharin | 0.050 | Saccharin | 0.050 |
| 100% | 99.257 | 100% | 99.065 | 100% Ethanol | 98.617 |
| Ethanol | | Ethanol | | | |
| Alpha- | 0.240 | Alpha- | 0.432 | Alpha- | 0.880 |
| tocopherol | | tocopherol | | tocopherol | |
| (5 mM) | | (10 mM) | | (20 mM) | |

### Example Formulation 29 (Butylated hydroxytoluene, pH 5.0)

| Material | % w/w |
|---|---|
| Buprenorphine | 0.253 |
| Levomenthol | 0.200 |
| Saccharin | 0.050 |
| 100% Ethanol | 99.447 |
| Butylated hydroxytoluene (BHT) | 0.050 |
| (2.27 mM) | |

### Example Formulation 30 (Butylated hydroxyanisole, pH 5.0)

| Material | % w/w |
|---|---|
| Buprenorphine | 0.253 |
| Levomenthol | 0.200 |
| Saccharin | 0.050 |
| 100% Ethanol | 99.447 |
| Butylated hydroxyanisole (BHA) | 0.050 |
| (2.77 mM) | |

The following example formulations may be prepared for comparison with Comparator Formulation A

### Example Formulation 31 (Ascorbic Acid)

| a) | | b) | | c) | |
|---|---|---|---|---|---|
| Material | % w/w | Material | % w/w | Material | % w/w |
| Bupre- | 0.253 | Bupre- | 0.253 | Buprenorphine | 0.253 |
| norphine | | norphine | | | |
| Levomenthol | 0.200 | Levomenthol | 0.200 | Levomenthol | 0.200 |
| Saccharin | 0.450 | Saccharin | 0.450 | Saccharin | 0.450 |
| Sodium | | Sodium | | Sodium | |
| 100% | 99.010 | 100% | 98.92 | 100% Ethanol | 98.740 |
| Ethanol | | Ethanol | | | |
| Ascorbic | 0.087 | Ascorbic | 0.177 | Ascorbic acid | 0.357 |
| acid (5 mM) | | acid | | (20 mM) | |
| | | (10 mM) | | | |

a) pH 6.2
b) pH 5.9
c) pH 5.7

### Example Formulation 32 (Alpha-tocopherol, pH 8.5)

| a) | | b) | | c) | |
|---|---|---|---|---|---|
| Material | % w/w | Material | % w/w | Material | % w/w |
| Bupre- | 0.253 | Bupre- | 0.253 | Buprenorphine | 0.253 |
| norphine | | norphine | | | |
| Levomenthol | 0.200 | Levomenthol | 0.200 | Levomenthol | 0.200 |
| Saccharin | 0.450 | Saccharin | 0.450 | Saccharin | 0.450 |
| Sodium | | Sodium | | Sodium | |
| 100% | 98.857 | 100% | 98.665 | 100% Ethanol | 98.217 |
| Ethanol | | Ethanol | | | |
| Alpha- | 0.240 | Alpha- | 0.432 | Alpha- | 0.880 |
| tocopherol | | tocopherol | | tocopherol | |
| (5 mM) | | (10 mM) | | (20 mM) | |

### Example Formulation 33 (Butylated hydroxytoluene, pH 8.5)

| Material | % w/w |
|---|---|
| Buprenorphine | 0.253 |
| Levomenthol | 0.200 |
| Saccharin Sodium | 0.450 |
| 100% Ethanol | 99.047 |
| Butylated hydroxytoluene (BHT) | 0.050 |

### Example Formulation 34 (Butylated hydroxyanisole, pH 8.5)

| Material | % w/w |
|---|---|
| Buprenorphine | 0.253 |
| Levomenthol | 0.200 |
| Saccharin Sodium | 0.450 |
| 100% Ethanol | 99.047 |
| Butylated hydroxyanisole (BHA) | 0.050 |
| (2.77 mM) | |

### Example Formulation 35 (Butylated hydroxyanisole, pH 6.5)

| Material | % w/w |
|---|---|
| Buprenorphine | 5.06 |
| Levomenthol | 0.200 |
| Saccharin Sodium | 0.450 |
| 100% Ethanol | 99.047 |
| Butylated hydroxyanisole (BHA) | 0.050 |

### Example Formulation 36 (Butylated hydroxytoluene, pH 6.6)

| Material | % w/w |
|---|---|
| Buprenorphine | 5.06 |
| Levomenthol | 0.200 |
| Saccharin | 0.05 |
| 100% Ethanol | 89.69 |
| Butylated hydroxytoluene (BHT) | 5.00 |

### Example Formulation 37 (Butylated hydroxytoluene, pH 6.5)

| Material | % w/w |
|---|---|
| Buprenorphine | 5.06 |
| Levomenthol | 0.200 |
| Saccharin | 0.05 |
| 100% Ethanol | 92.69 |
| Butylated hydroxytoluene (BHT) | 2.00 |

Compositions were prepared as follows:

### Comparator Formulation A

2.9729 kg of absolute ethanol was weighed into a 7 litre stainless steel container. Buprenorphine base (0.0076 kg) was added to the ethanol and mixed using a mobile stirrer until completely dissolved. Levomenthol (0.0060 kg) was added to the solution and mixed using a mobile stirrer until completely dissolved. Saccharin sodium (0.0135) was added to the solution and mixed using a mobile stirrer until completely dissolved.

### Comparator Formulation B

2.9849 kg of absolute ethanol was weighed into a 7 litre stainless steel container. Buprenorphine base (0.0076 kg) was added to the ethanol and mixed using a mobile stirrer until completely dissolved. Levomenthol (0.0060 kg) was added to the solution and mixed using a mobile stirrer until completely dissolved. Saccharin (0.0015) was added to the solution and mixed using a mobile stirrer until completely dissolved.

### Example Formulation 27c

2.9742 kg of absolute ethanol was weighed into a 7 litre stainless steel container. Buprenorphine base (0.0076 kg) was added to the ethanol and mixed using a mobile stirrer until completely dissolved. Ascorbic acid (0.0107 Kg) was added to the solution and mixed using a mobile stirrer until completely dissolved. Levomenthol (0.0060 kg) was added to the solution and mixed using a mobile stirrer until completely dissolved. Saccharin (0.0015) was added to the solution and mixed using a mobile stirrer until completely dissolved.

All of Examples 27-30 were prepared by an analogous procedure.

### Example Formulation 31c

2.9622 kg of absolute ethanol was weighed into a 7 litre stainless steel container. Buprenorphine base (0.0076 kg) was added to the ethanol and mixed using a mobile stirrer until completely dissolved. Ascorbic acid (0.0107 Kg) was added to the solution and mixed using a mobile stirrer until completely dissolved. Levomenthol (0.0060 kg) was added to the solution and mixed using a mobile stirrer until completely dissolved. Saccharin Sodium (0.0135 Kg) was added to the solution and mixed using a mobile stirrer until completely dissolved.

All of Examples 31-37 were prepared by an analogous procedure.

### Results

### 1. Bioavailability Clinical Trials Data

A single dose, randomised crossover study to compare the rate and extent of absorption of three formulations of buprenorphine in healthy male volunteers was carried out.

A single dose of buprenorphine was administered sublingually to each volunteer. In each study period, 14 blood collections were made over 10 hours, at the following times: immediately before administration of the study drug (0.0 hours) and at 0.25, 0.5, 0.75, 1.0, 1.33, 1.67, 2.0, 2.5, 3.0, 4.0, 6.0, 8.0 and 10.0 hours after administration. The total volume of blood taken from each volunteer (including screening and post study) was approximately 300 ml. Plasma concentration of buprenorphine was measured.

15 healthy male volunteers participated in the study; 4 withdrew before completion and 11 completed all three periods of the study. The interval between each of the periods was one week.

The study was carried out using the following treatments:
Treatment A=Buprenorphine 0.4 mg spray Formulation A (pH 8.5), 1*0.4 mg spray, Pharmasol Ltd., UK
Treatment B=Buprenorphine 0.4 mg spray Formulation B (pH 5.0), 1*0.4 mg spray, Pharmasol Ltd., UK
Treatment C=Temgesic® SL tablets, 1*0.4 mg sub-lingual tablet, Schering Plough, UK

The results are shown graphically in FIG. 3.

Below is a table summarising the results:

TABLE 1

| | Mean Buprenorphine plasma concentration | | |
|---|---|---|---|
| | (ng/ml) | | |
| Time | Treatment A | Treatment B | Treatment C |
| 0 mins | 0.000 | 0.089 | 0.018 |
| 15 mins | 0.057 | 0.054 | 0.037 |
| 30 mins | 0.213 | 0.238 | 0.150 |
| 45 mins | 0.387 | 0.382 | 0.231 |
| 1 hr | 0.444 | 0.405 | 0.302 |
| 1 hr 20 mins | 0.460 | 0.495 | 0.290 |
| 1 hr 40 mins | 0.537 | 0.532 | 0.375 |
| 2 hrs | 0.534 | 0.569 | 0.333 |
| 2 hrs 30 mins | 0.493 | 0.515 | 0.289 |
| 3 hrs | 0.460 | 0.459 | 0.281 |
| 4 hrs | 0.373 | 0.418 | 0.253 |
| 6 hrs | 0.202 | 0.213 | 0.118 |
| 8 hrs | 0.132 | 0.132 | 0.059 |
| 10 hrs | 0.089 | 0.092 | 0.031 |

The two spray formulations (Treatment A, Buprenorphine spray 0.4 mg Formulation A (pH 8.5), and Treatment B, Buprenorphine spray 0.4 mg Formulation B (pH 5.0), Pharmasol Ltd., UK) have a similar rate and extent of absorption.

Treatment A and Treatment B were suprabioavailable compared to the tablet formulation (Treatment C, Temgesic® SL tablets 0.4 mg, Schering Plough, UK).

### 2. HPLC Comparison of Degradation of Comparator Formulations A and B

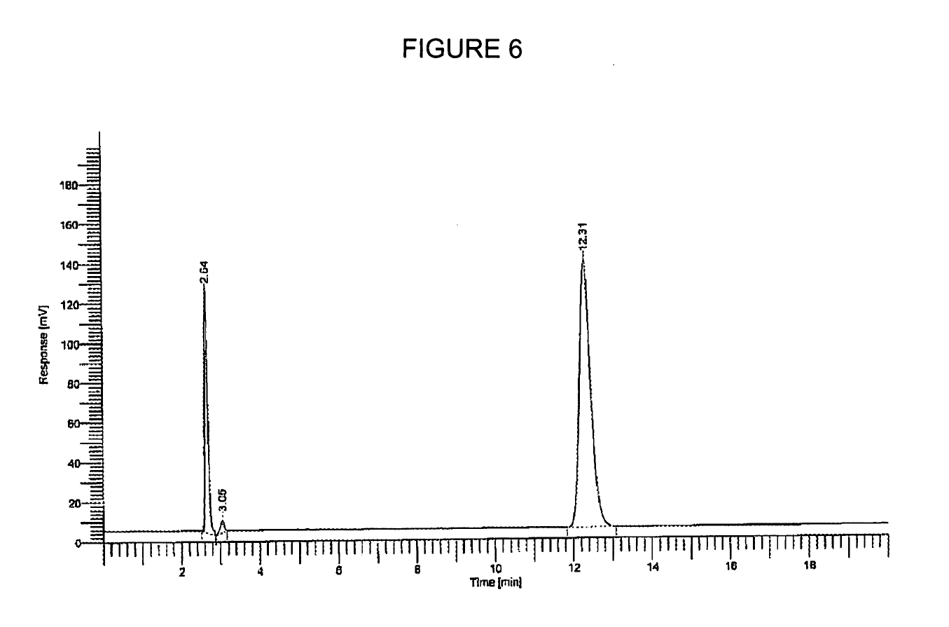

FIGS. 4 and 6 show the HPLC trace of comparator formulations A and B at the initial time point. As can clearly be seen from FIGS. 4 and 6, there an HPLC peak corresponding to buprenorphine at the initial time point.

In FIG. 4, peaks at 2.64 and 3.05 mins are injection peaks and the peak at 12.26 mins is the buprenorphine peak. In FIG. 6, peaks at 2.64 and 3.05 mins are injection peaks and the peak at 12.31 mins is the buprenorphine peak.

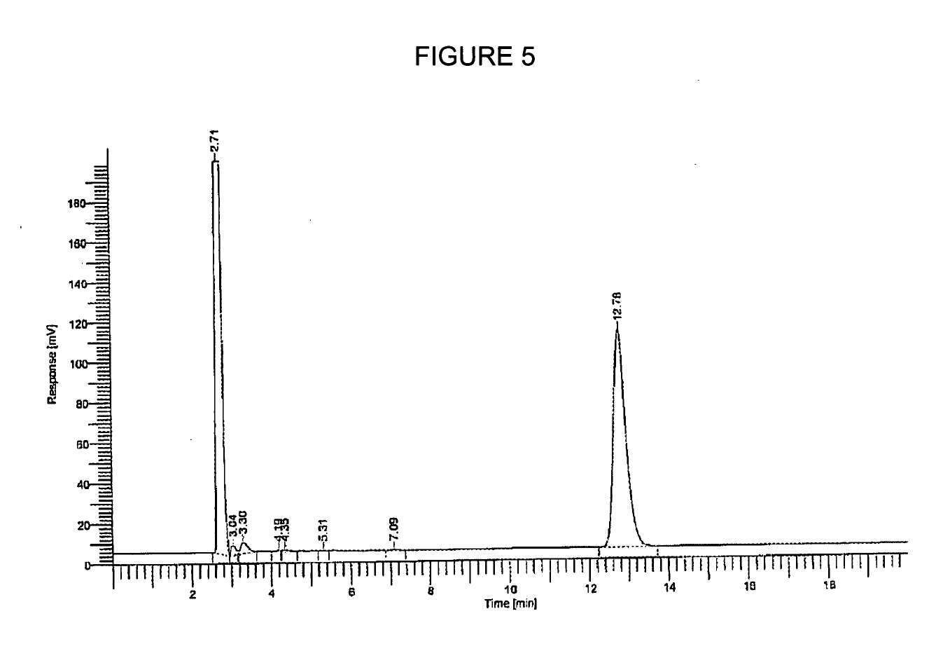

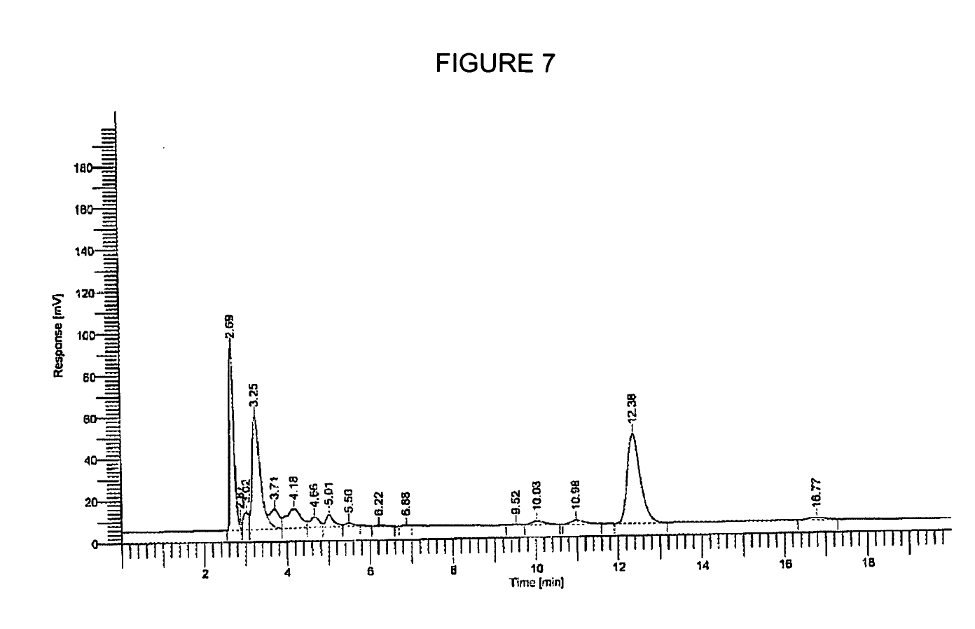

FIGS. 5 and 7 show the HPLC trace of comparator formulations A and B, 6 months after the initial time point, after degradation has occurred. As can clearly be seen from FIGS. 5 and 7, a number of new peaks corresponding to degradation products, which were not present at the initial time point, have developed.

In FIG. 5, degradation peaks are observed at 3.30, 4.19, 4.35, 5.31, 7.09 mins. In FIG. 7, degradation peaks are observed at 3.25, 3.71, 4.18, 4.66, 5.01, 5.50, 6.22, 6.88, 9.52, 10.03, 10.98, 16.77 mins.

Comparison of FIG. 5 with FIG. 7 clearly shows that more peaks of greater intensity are present after 6 months in the spectrum of comparator formulation B than comparator formulation A.

### 2. LCMS

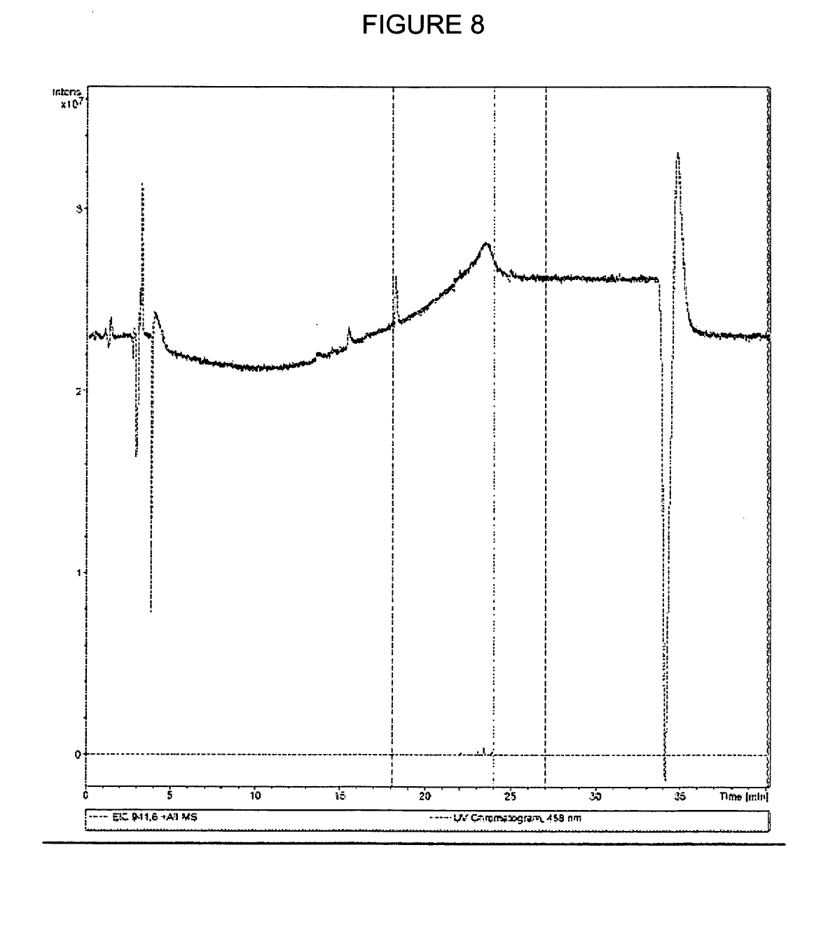

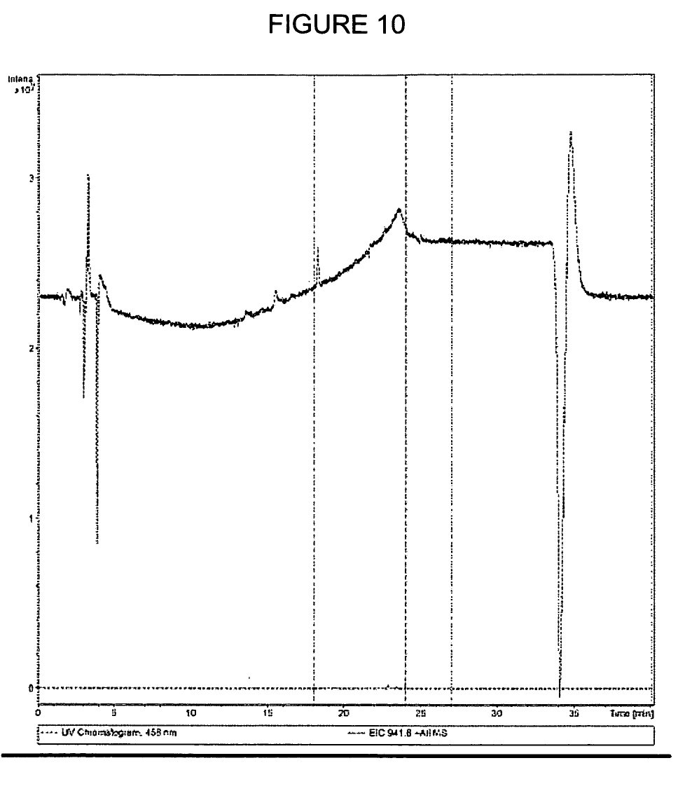

FIGS. 8 and 10 show the LCMS trace of comparator formulations A and B at the initial time point. As can clearly be seen from FIGS. 8 and 10, there is no LCMS m/z peak corresponding to the dimer or a UV-vis absorption corresponding to the dimer at the initial time point.

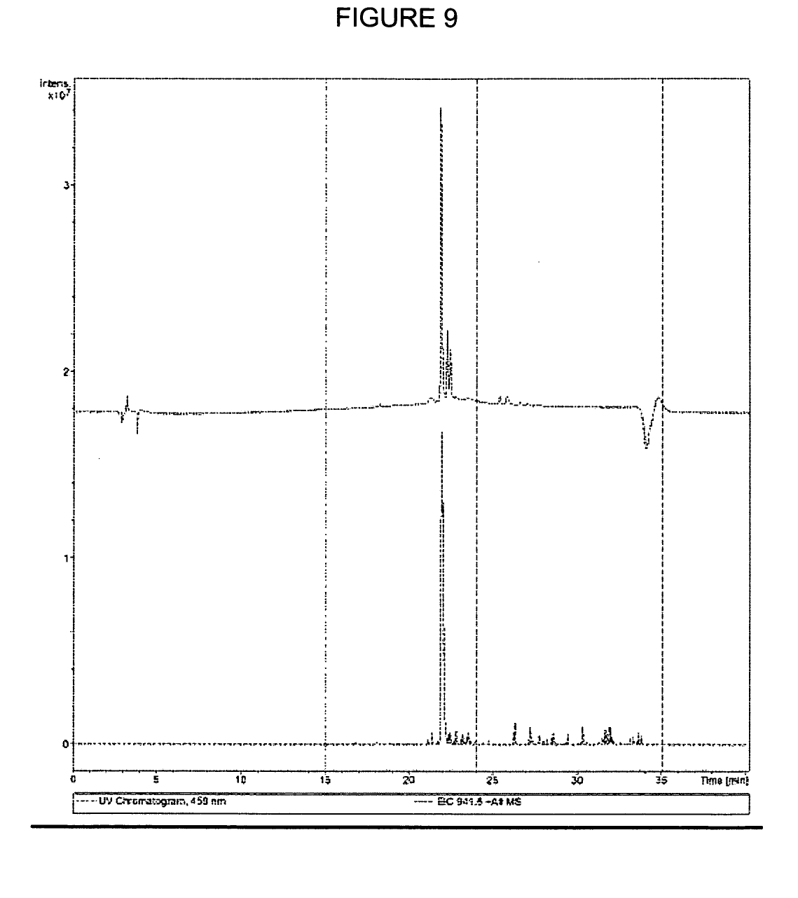

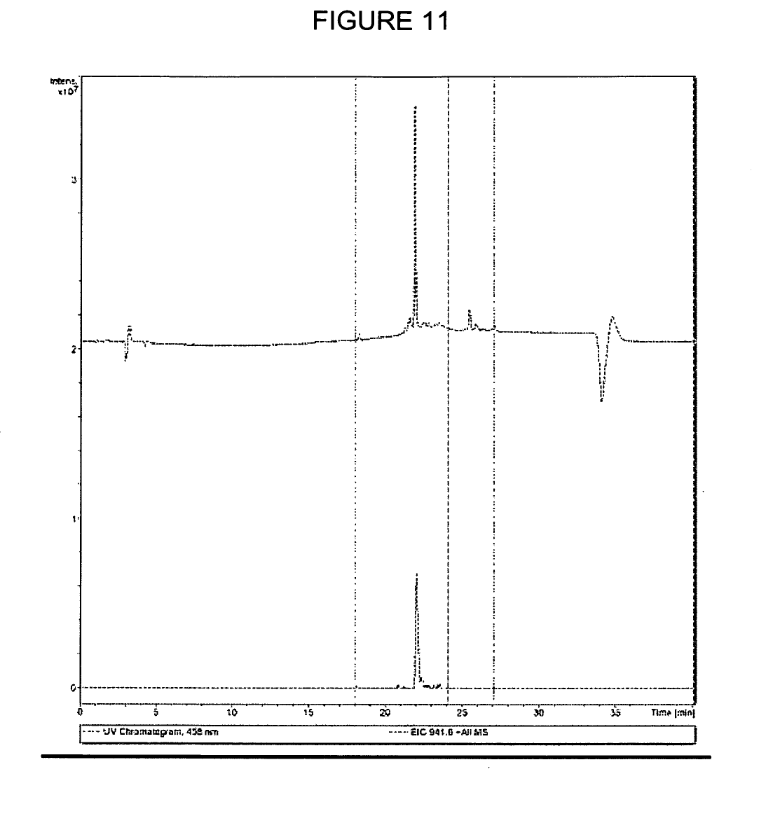

FIGS. 9 and 11 show the LCMS trace of comparator formulations A and B, 12 months after the initial time point, after degradation has occurred. As can clearly be seen from FIGS. 9 and 11, an m/z peak corresponding to the dimer (at m/z 941.6) and a UV-vis absorption (at 458 nm) corresponding to the dimer, which were not present at the initial time point, have developed.

### 3. UV-vis Scan

All buprenorphine formulations were made and were initially tested for degradation, assay for buprenorphine and scanned on the UV-vis spectrometer, before being put on stability at 40° C./75% RH. At each time point, a sample was removed from each formulation and scanned on the UV-vis spectrometer. After the scan was performed, the sample was disposed of.

The UV-vis scan was performed at 458 nm, as this is the optimum wavelength to detect the buprenorphine dimerisation. For the time points at 3, 4 and 6 weeks, a scan between 700-200 nm was performed on the same sample in addition to the scan at 458 nm.

### Buprenorphine Formulations+Ascorbic Acid/Alpha-Tocopherol

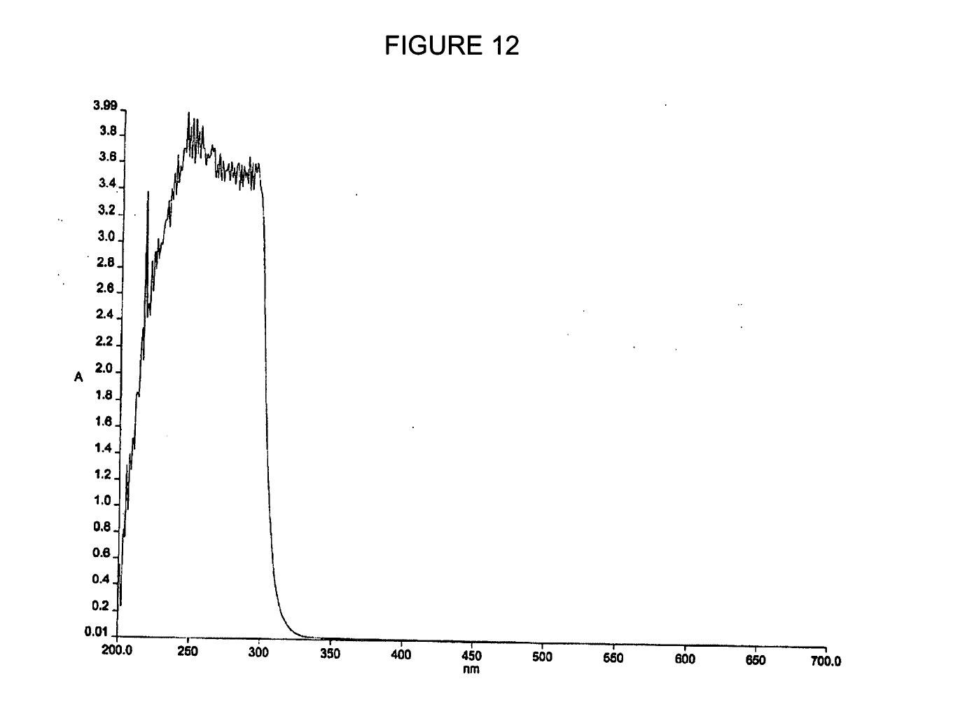

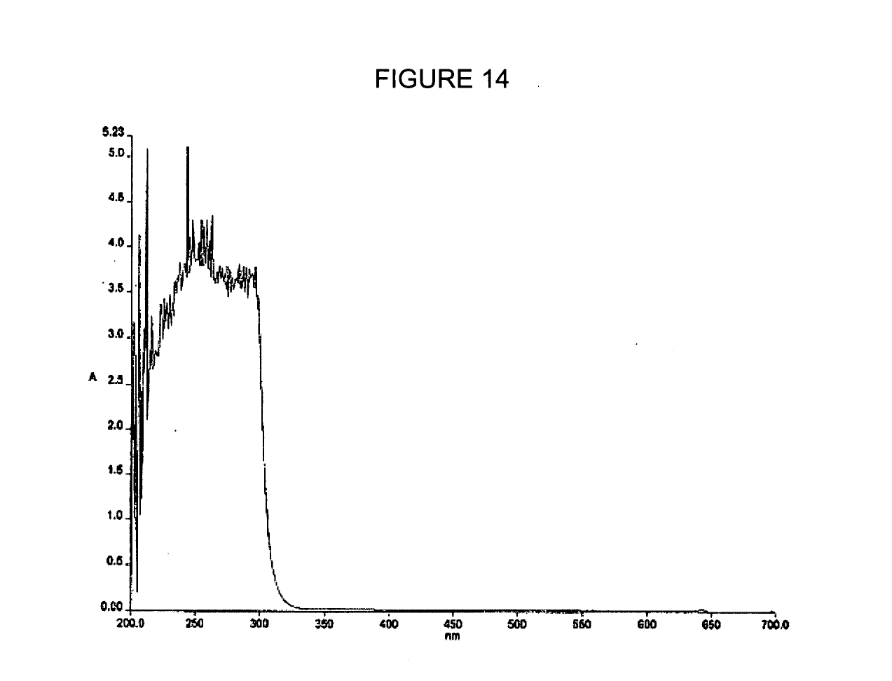

FIGS. 12 and 14 show that at the initial time point there was no UV-vis absorption between 400 nm and 500 nm.

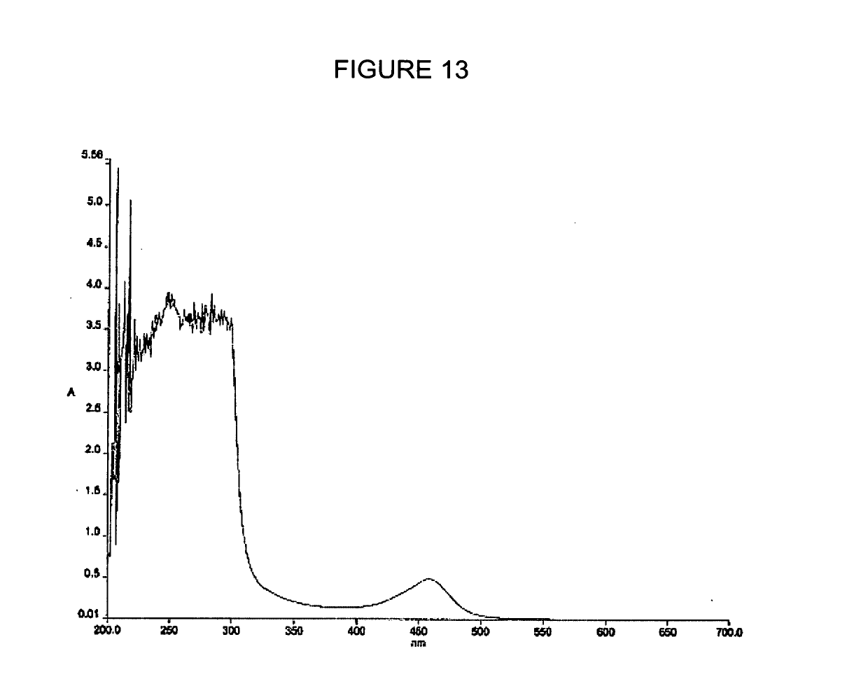

FIG. 13 shows that for formulation B after 6 weeks, a peak between 400 nm and 500 nm is clearly visible. This peak corresponds to the dimerisation product.

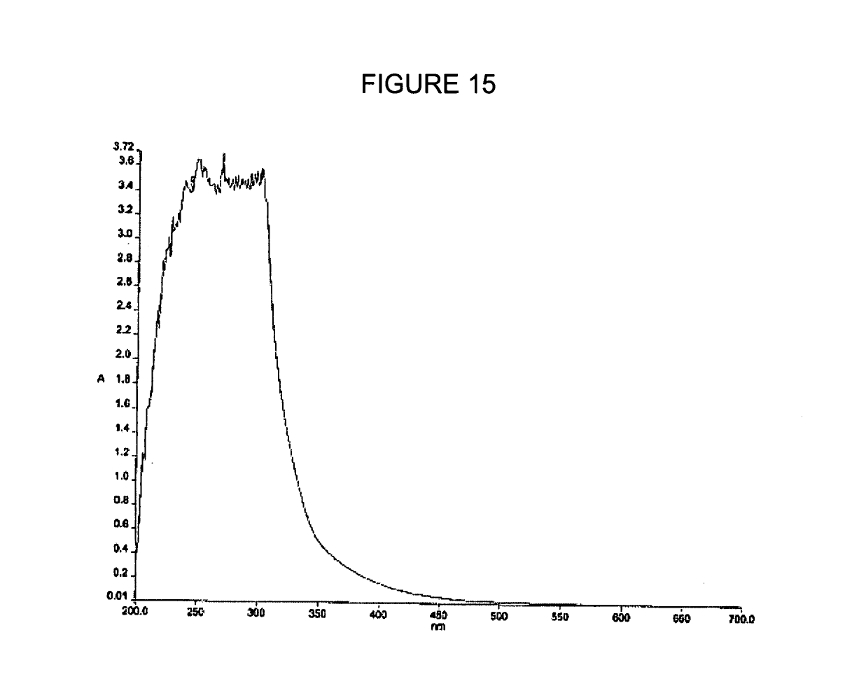

FIG. 15 shows that for Example 27c after 6 weeks, the presence of 20 mM ascorbic acid has prevented the development of a peak between 400 nm and 500 nm i.e. the presence of antioxidant in Example 27c has inhibited the dimerisation process of buprenorphine.

Below is a table summarising the UV-vis results for the formulations including ascorbic acid and alpha-tocopherol.

TABLE 2

| | | UV-vis scan @ 458 nm | | | | | |
|---|---|---|---|---|---|---|---|
| | | | 1 | 2 | 3 | 4 | 6 |
| | | Initial | week | weeks | weeks | weeks | weeks |
| Comparator | Blank | 0.0129 | 0.0536 | 0.1295 | 0.2516 | 0.6695 | 0.4954 |
| formulation B | | | | | | | |
| (no antioxidants | | | | | | | |
| added) | | | | | | | |
| Example 27a | 5 mM | 0.0122 | 0.0081 | 0.0242 | 0.0121 | 0.0159 | 0.0220 |
| | ascorbic | | | | | | |
| | acid | | | | | | |
| Example 27b | 10 mM | 0.0188 | 0.0194 | 0.0282 | 0.0733* | 0.0719* | 0.0308 |
| | ascorbic | | | | | | |
| | acid | | | | | | |
| Example 27c | 20 mM | 0.0152 | 0.0191 | 0.0329 | 0.0224 | 0.1819* | 0.0486 |
| | ascorbic | | | | | | |
| | acid | | | | | | |
| Example 28a | 5 mM | 0.0216 | 0.0140 | 0.0241 | 0.0248 | 0.0602 | nd |
| | alpha- | | | | | | |
| | tocopherol | | | | | | |
| Example 28b | 10 mM | 0.0131 | 0.0173 | 0.0222 | 0.0374 | 0.0575 | nd |
| | alpha- | | | | | | |
| | tocopherol | | | | | | |
| Example 28c | 20 mM | 0.0272 | 0.0218 | 0.0349 | 0.0520 | 0.0989 | nd |
| | alpha- | | | | | | |
| | tocopherol | | | | | | |

nd = not determined

*These anomalous results from the scan at 458 nm were abnormally high. In addition to the scan at 458 nm, a scan between 700-200 nm for the same sample was also performed at this time point. The scan between 700-200 nm showed that there was not a peak around 458 nm. The reason for the high reading in the 458 nm scan is the calibration of the equipment (for the anomalous results, the lowest possible reading was considerably higher than that for the other samples).

After 6 weeks the ascorbic acid formulations appear colourless to the naked eye. By contrast the alpha-tocopherol and blank formulations have a yellow tinge compared to the comparator formulation suggesting that ascorbic acid has a greater effect on enhancing stability than does alpha-tocopherol.

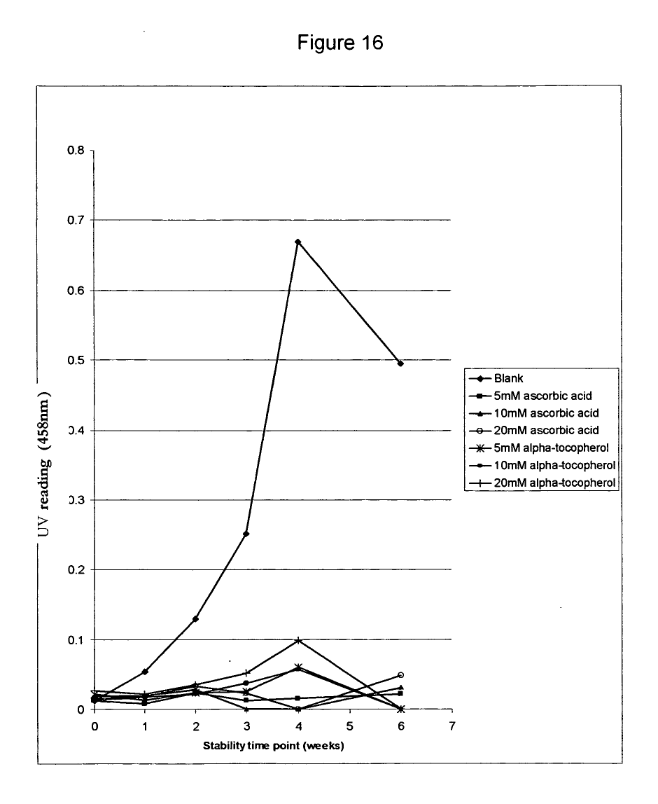

The results of Table 2 are shown graphically in FIG. 16.

FIG. 16 clearly shows that ascorbic acid and alpha-tocopherol significantly enhance stability of the buprenorphine.

### Buprenorphine Formulations+BHT/BHA

Below is a table summarising the UV-vis results for the formulations including BHT and BHA

TABLE 3

| | | UV-vis scan @ 458 nm | | |
|---|---|---|---|---|
| | | 1 week | 4 weeks | 5 weeks |
| Comparator formulation B | Blank | 0.0044 | 0.0977 | 0.0973 |
| (no antioxidants added) | | | | |
| Example 29 | BHT 0.05% | 0.0000 | 0.0130 | −0.0230 |
| Example 30 | BHA 0.05% | 0.0029 | 0.0119 | −0.0200 |

Bold = Although these results suggest that there has been no colour change, scans between 700-200 nm show a peak appearing, suggesting dimerisation.

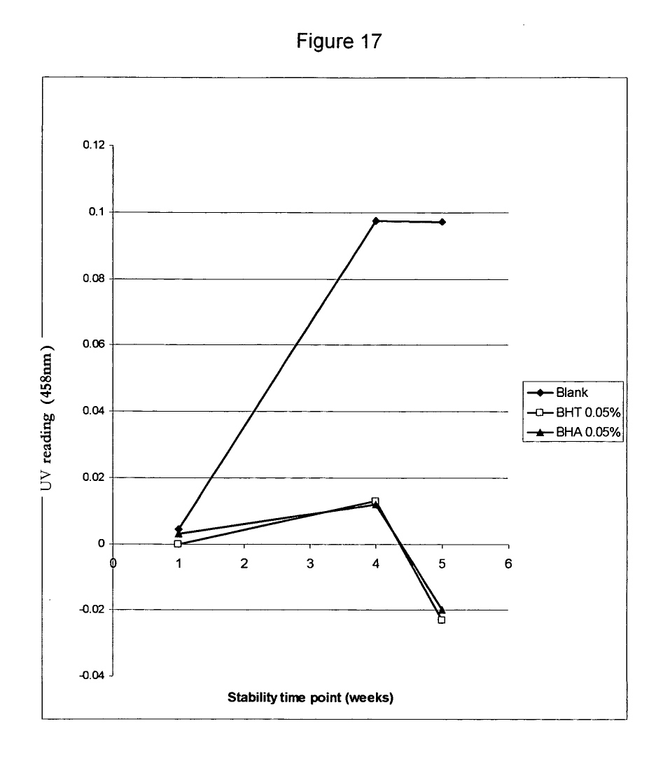

The results of Table 3 are shown graphically in FIG. 17.

FIG. 17 clearly shows that BHT and BHA make a significantly positive difference on the stability of the buprenorphine.

### 4. Degradation Assay

The degradation and assay for buprenorphine tests were carried out as per methods used for standard productions to see if the antioxidants had any effect on the buprenorphine formulation. The data for the following tables was generated from spectroscopic data.

Buprenorphine Formulations+ascorbic acid/alpha-tocopherol

Below is a table summarising the degradation results for the formulations containing ascorbic acid and alpha-tocopherol.

TABLE 4

| | | HPLC degradation | | | |
|---|---|---|---|---|---|
| | | Initial | 3 weeks | 5 weeks | 6 weeks |
| Comparator | Blank | 0.0 | 0.0066 | 0.0177 | 0.0135 |
| formulation B | | | | | |
| (no antioxidants | | | | | |
| added) | | | | | |
| Example 27a | 5 mM ascorbic | 0.0 | 0.0014 | 0.0054 | 0.0024 |
| | acid | | | | |
| Example 27b | 10 mM | 0.0 | nd | 0.0054 | 0.0043 |
| | ascorbic acid | | | | |
| Example 27c | 20 mM | 0.0 | 0.0027* | 0.0000 | 0.0000 |
| | ascorbic acid | | | | |
| Example 28a | 5 mM alpha- | 0.0 | 0.0000 | nd | 0.0007 |
| | tocopherol | | | | |
| Example 28b | 10 mM alpha- | 0.0 | nd | nd | 0.0007 |
| | tocopherol | | | | |
| Example 28c | 20 mM alpha- | 0.0 | 0.0005 | nd | 0.0005 |
| | tocopherol | | | | |

nd = not determined
Specification: degradent < 0.0005%
Bold = Out of spec
*thought to be an anomaly, as shown by the results in weeks 5 & 6.

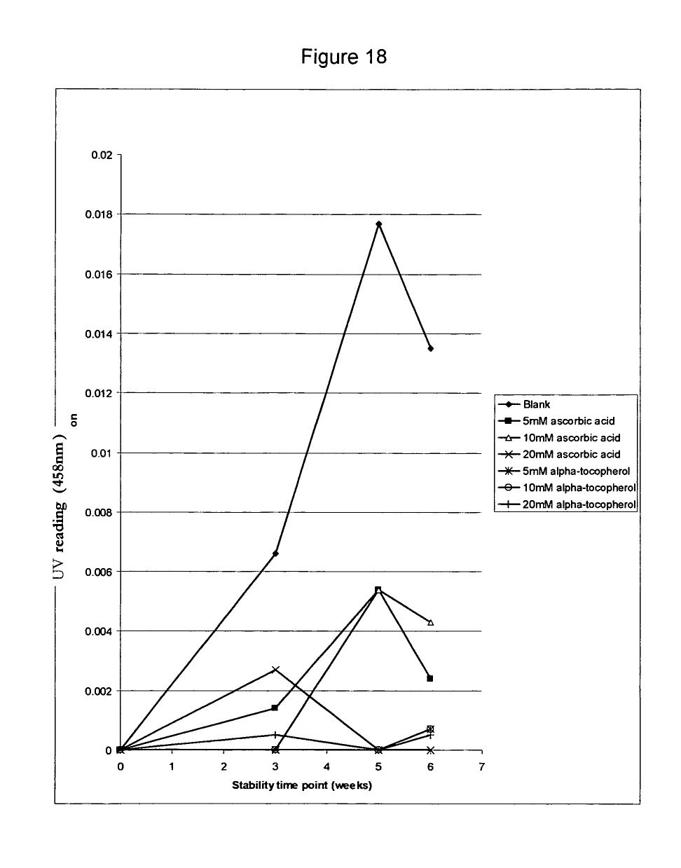

The results of Table 4 are shown graphically in FIG. 18.

FIG. 18 clearly shows that ascorbic acid and alpha-tocopherol control the level of degradation compared to the comparator formulation of buprenorphine.

Degradation results from stability trial: Comparator Formulation A after 1 month:

TABLE 5

| | | HPLC degradation |
|---|---|---|
| Formulation | Time and temperature | (% w/v) |
| Comparator Formulation A | 1 month @ 30° C. | 0.0021 |
| (no antioxidant) | | |
| Comparator Formulation A | 1 month @ 40° C. | 0.0068 |
| (no antioxidant) | | |

The Comparator Formulation A used in this stability trial over 1 month at 30° C. and 40° C. was taken from a 3 litre manufactured batch of said formulation.

Degradation results from stability trial: Comparator Formulation B after 1 month:

TABLE 6

| | | HPLC degradation |
|---|---|---|
| Formulation | Time and temperature | (% w/v) |
| Comparator Formulation B | 1 month @ 30° C. | 0.00 |
| (no antioxidant) | | |
| Comparator Formulation B | 1 month @ 40° C. | 0.0002 |
| (no antioxidant) | | |

The Comparator Formulation B used in this stability trial over 1 month at 30° C. and 40° C. was taken from a 3 litre manufactured batch of said formulation

### Buprenorphine Formulations+BHT/BHA

Below is a table summarising the degradation results for the formulations containing BHT and BHA.

TABLE 7

| | | HPLC degradation | | |
|---|---|---|---|---|
| | | Initial | 1 week | 5 weeks |
| Comparator formulation B | Blank | 0.00 | 0.0015 | 0.0095 |
| (no antioxidants added) | | | | |
| Example 29 | BHT 0.05% | 0.0 | 0.0 | 0.0013 |
| Example 30 | BHA 0.05% | 0.0 | 0.0 | 0.0016 |

Specification: degradent < 0.0005%
Bold = Out of spec

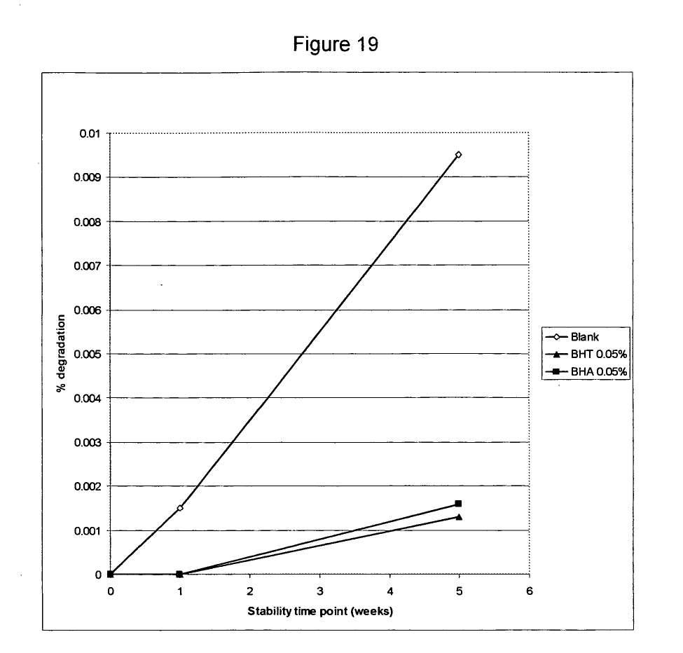

The results of Table 7 are shown graphically in FIG. 19.

FIG. 19 clearly shows that the formulations containing BHT and BHA keep the level of degradation at a better level compared to the comparator formulation of buprenorphine.

### 5. Buprenorphine Assay

The data for the following tables was generated from spectroscopic data.

Buprenorphine Formulations+ascorbic acid/alpha-tocopherol

Below is a table summarising the results for the assay for buprenorphine for the formulations containing ascorbic acid and alpha-tocopherol.

TABLE 8

| | | HPLC Buprenorphine assay | | |
|---|---|---|---|---|
| | | 3 weeks | 5 weeks | 6 weeks |
| Comparator formulation B | Blank | 0.21 | 0.20 | 0.19 |
| (no antioxidants added) | | | | |
| Example 27a | 5 mM | 0.20 | 0.21 | 0.21 |
| | ascorbic acid | | | |
| Example 27b | 10 mM | nd | 0.21 | 0.21 |
| | ascorbic acid | | | |
| Example 27c | 20 mM | 0.20 | 0.21 | 0.21 |
| | ascorbic acid | | | |
| Example 28a | 5 mM alpha- | 0.21 | nd | 0.21 |
| | tocopherol | | | |
| Example 28b | 10 mM alpha- | nd | nd | 0.20 |
| | tocopherol | | | |
| Example 28c | 20 mM alpha- | 0.21 | nd | 0.21 |
| | tocopherol | | | |

nd = not determined

### Buprenorphine Formulations+BHT/BHA

Below is a table summarising the results for the assay for buprenorphine for the formulations containing BHT and BHA.

TABLE 9

| | | HPLC | | |
|---|---|---|---|---|
| | | buprenorphine assay | | |
| | | Initial | 1 week | 5 weeks |
| Comparator formulation B | Blank | nd | 0.21 | 0.20 |
| (no antioxidants added) | | | | |
| Example 29 | BHT 0.05% | 0.21 | 0.21 | 0.21 |
| Example 30 | BHA 0.05% | 0.21 | 0.21 | 0.21 |

nd = not determined

In summary, the tables show that the antioxidants have no negative effect on the assay for buprenorphine.

### 6. 2 Month Stability Analysis

A 2 month stability trial was conducted at 40° C. and at 75% relative humidity. At various time points (i.e. initial, 2 week, 1 month and 2 months), the test formulations were measured for appearance of colour and a UV scan (to detect possible dimerisation), buprenorphine assay, pH and the presence of related substances.

### Buprenorphine Formulation (Control)

Below is a table summarising the stability results for a formulation containing 40 mg/ml buprenorphine in the absence of antioxidant as control

TABLE 10

| | Initial | 2 weeks | 1 month | 2 months |
|---|---|---|---|---|
| Appearance | Conforms | Conforms | Yellow | Yellow |
| Assay | 4.036 | 4.077 | 4.066 | 4.003 |
| Buprenorphine | | | | |
| Related | 0.0 | n/d | 1.3, 8 × <0.5% | 0.6, 7 × <0.5% |
| substances | | | | |
| Total rel subs | 0.0 | n/d | 3.1 | 1.3 |
| pH | 7.39 | 7.40 | 7.58 | 7.31 |
| UV scan | 0.0317 | 0.092 | 0.1946 | 0.4502 |
| @548 nm | | | | |

Bold = out of spec

### Buprenorphine Formulation−BHT

Below is a table summarising the stability results for a formulation containing 40 mg/ml buprenorphine in the presence of 2% butylated hydroxytoluene (BHT).

TABLE 11

| | Initial | 2 weeks | 1 month | 2 months |
|---|---|---|---|---|
| Appearance | Conforms | Conforms | Conforms | Yellow tinge |
| Assay | 4.026 | 4.079 | 4.065 | 4.040 |
| Buprenorphine | | | | |
| Related | 0.0 | n/d | 7 × <0.5% | 7 × <0.5% |
| substances | | | | |
| Total rel subs | 0.0 | n/d | 0.8 | 0.9 |
| pH | 7.02 | 7.03 | 7.29 | 7.18 |
| UV scan | 0.0359 | 0.0282 | 0.0478 | 0.1718 |
| @548 nm | | | | |

Bold = out of spec

Below is a table summarising the stability results for a formulation containing 40 mg/ml buprenorphine in the presence of 5% butylated hydroxytoluene (BHT).

TABLE 12

| | Initial | 2 weeks | 1 month | 2 months |
|---|---|---|---|---|
| Appearance | Conforms | Conforms | Conforms | Yellow tinge |
| Assay | 4.091 | 4.113 | 4.105 | 4.108 |
| Buprenorphine | | | | |
| Related | 0.0 | n/d | 9 × 0.5% | 8 × 0.5% |
| substances | | | | |
| Total rel subs | 0.0 | n/d | 1.1 | 1.2 |
| pH | 7.15 | 7.30 | 7.33 | 7.20 |
| UV scan | 0.0526 | 0.0337 | 0.0557 | 0.1278 |
| @548 nm | | | | |

Bold = out of spec

### Buprenorphine Formulation+BHA

Below is a table summarising the stability results for a formulation containing 40 mg/ml buprenorphine in the presence of 2% butylated hydroxyanisole (BHA).

TABLE 13

| | Initial | 2 weeks | 1 month | 2 months |
|---|---|---|---|---|
| Appearance | Conforms | Conforms | Yellow | Yellow |
| Assay | 4.041 | 4.063 | 4.104 | 4.088 |
| Buprenorphine | | | | |
| Related | 0.0 | n/d | Out of spec | Out of spec |
| substances | | | | |
| Total rel subs | 0.0 | n/d | Out of spec | Out of spec |
| pH | 6.92 | 7.00 | 7.43 | 7.1 |
| UV scan | 0.0864 | 0.1013 | 0.1636 | 0.3252 |
| @548 nm | | | | |

Bold = out of spec

Below is a table summarising the stability results for a formulation containing 40 mg/ml buprenorphine in the presence of 5% butylated hydroxyanisole (BHA).

TABLE 14

| | Initial | 2 weeks | 1 month | 2 months |
|---|---|---|---|---|
| Appearance | Conforms | Conforms | Yellow | Yellow |
| Assay | 4.055 | 4.137 | 4.142 | 4.145 |
| Buprenorphine | | | | |
| Related | 0.0 | n/d | Out of spec | Out of spec |
| substances | | | | |
| Total rel subs | 0.0 | n/d | Out of spec | Out of spec |
| pH | 6.92 | 7.02 | 7.59 | 7.33 |
| UV scan | 0.0556 | 0.1149 | 0.1998 | 0.3746 |
| @548 nm | | | | |

Bold = out of spec

In summary, the tables show that the antioxidants BHT and BHA have a positive effect upon the stability of buprenorphine when compared with the results obtained in the absence of antioxidant. In particular, BHT appeared to confer optimal stability upon buprenorphine.

7. Comparative Stability Analysis with Propyl Gallate as Antioxidant

A stability trial using propyl gallate as antioxidant was conducted in an analogous manner to that conducted in section 6 above.

Below is a table summarising the stability results for a formulation containing 40 mg/ml buprenorphine in the presence of 2% propyl gallate.

TABLE 15

| | Initial | 2 weeks |
|---|---|---|
| Appearance | Conforms | Yellow |
| Assay | 4.133 | 4.170 |
| Buprenorphine | | |
| Related | 0.0 | 5 × <0.5% |
| substances | | |
| Total rel subs | 0.0 | 0.9 |
| pH | 7.54 | 7.26 |
| UV scan | 0.0675 | 0.2048 |
| @548 nm | | |

Bold = out of spec

Below is a table summarising the stability results for a formulation containing 40 mg/ml buprenorphine in the presence of 5% propyl gallate.

TABLE 16

| | Initial | 2 weeks |
|---|---|---|
| Appearance | Conforms | Yellow |
| Assay | 4.174 | 4.259 |
| Buprenorphine | | |
| Related | 0.0 | 0.7, 3 × <0.5% |
| substances | | |
| Total rel subs | 0.0 | 1.0 |
| pH | 7.39 | 7.59 |
| UV scan | 0.0872 | 0.2063 |
| @548 nm | | |

Bold = out of spec

In summary, it would be appear that propyl gallate provides a negative effect upon the stability of buprenorphine when compared with the results obtained in section 6 in the absence of antioxidant.

### SUMMARY

Antioxidants have generally been shown to have a beneficial effect on the stability of buprenorphine formulations.

### Ascorbic Acid

Inclusion of ascorbic acid in the example formulations prevented the yellow colour from forming over six weeks, and hence stopped the dimerisation process. It also reduced the level of degradation products shown on the HPLC, with particular significance when looking at the 20 mM concentration, which keeps the degradation results in specification over a six week period.

### Alpha-Tocopherol

Inclusion of alpha-tocopherol in example formulations prevented the yellow colour from forming over three weeks, which is better than the comparator formulation but not as good as the ascorbic acid formulations. Reduction in degradation products as determined by HPLC appears to be better in the alpha-tocopherol formulations than in the ascorbic acid formulations.

### BHT and BHA

The degradation profile as determined by HPLC and the dimerisation results as determined by UV-vis are superior to those of the comparator formulation. The presence of BHT in particular was also seen to confer excellent stability results upon buprenorphine even after two months when stored at 40° C. and at 75% relative humidity.

### Propyl Gallate

The stability assessment demonstrated that propyl gallate provides a negative effect upon the stability of buprenorphine when present as a sole antioxidant. For example, dimerisation occurred after just 2 weeks when compared with 1 month in the absence of antioxidant.

All patents and patent applications mentioned herein are incorporated by reference in their entirety.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

We claim:

1. A non-pressurised pharmaceutical liquid solution spray composition comprising:
(i) buprenorphine; and
(ii) a solvent comprising ethanol
characterised in that the composition is substantially free of chloride.

2. A composition according to claim 1 wherein the concentration of ethanol in the solvent is greater than 90% w/w.

3. A composition according to claim 1 which is substantially free of water.

4. A composition according to claim 2 wherein the concentration of ethanol in the solvent is around 100% w/w.

5. A composition according to claim 1 wherein the concentration of buprenorphine in the composition is around 0.05-12% w/v.

6. A composition according to claim 1 wherein the pH of the composition is between around 4 and 6.

7. A composition according to claim 6 which contains saccharin.

8. A composition according to claim 1 which comprises a flavouring agent selected from menthol, peppermint oil and mixtures thereof.

9. A composition according to claim 1 formulated for transmucosal administration as a spray.

10. A composition according to claim 9 wherein the transmucosal administration is sublingual administration.

11. A composition according to claim 1 wherein the buprenorphine is employed in the form of its base.

12. A non-pressurised pharmaceutical liquid solution spray composition which comprises
buprenorphine as base;
a solvent selected from ethanol and ethanol/water mixtures;
saccharin;
wherein the composition is substantially free of chloride; and wherein the pH of the composition is between around 4 and 6.

13. A composition according to claim 12 wherein the concentration of buprenorphine base is 0.1-4% w/v.

14. A composition according to claim 12 wherein the solvent is ethanol.

15. A composition according to claim 12 which comprises a flavouring agent selected from menthol, peppermint oil and mixtures thereof.

16. A composition according to claim 12 which comprises an anti-oxidant.

17. A non-pressurised pharmaceutical liquid solution spray composition which comprises:
buprenorphine as base at a concentration of 4% w/v or more;
a solvent selected from ethanol and ethanol/water mixtures;
citric acid;
wherein the composition is substantially free of chloride; and wherein the pH of the composition is between around 4 and 6.

18. A composition according to claim 17 which comprises saccharin.

19. A composition according to claim 17 wherein the solvent is ethanol.

20. A composition according to claim 17 which comprises a flavouring agent selected from menthol, peppermint oil and mixtures thereof.

21. A composition according to claim 17 which comprises an anti-oxidant.

22. A composition according to claim 12 formulated for transmucosal administration as a spray.

23. A composition according to claim 17 formulated for transmucosal administration as a spray.

24. A composition according to claim 22 wherein the transmucosal administration is sublingual administration.

25. A method for the treatment or prevention of opiate dependency or abuse or pain, the method comprising administering to a patient in need of such treatment an effective amount of a composition according to claim 1.

26. A sealed container containing a plurality of doses of a composition according to claim 1.

27. A container according to claim 26 which is made out of glass.

28. A metered dose dispensing system comprising a sealed container according to claim 26 fitted with a metering pump, an actuator and a channeling device.

29. A metered dose dispensing system according to claim 28 containing a metering chamber which is adapted for dispensation with the container in the upright orientation and wherein the metering chamber is in communication with the composition by means of a dip-tube.

30. A metered dose dispensing system according to claim 28 adapted for transmucosal administration of the composition as a spray.

31. A metered dose dispensing system according to claim 30 wherein the transmucosal administration is sublingual administration.

32. A process for preparation a composition according to claim 1 which comprises:
(a) taking buprenorphine as base and a solvent comprising ethanol optionally containing the other formulation ingredients and dissolving the buprenorphine in the solvent; or
(b) taking buprenorphine as base and a solvent comprising ethanol and dissolving the buprenorphine in the solvent, then adding the other formulation ingredients; or
(c) the process of (a) or (b) in which the pH of the solvent is adjusted once all the other formulation ingredients are mixed together.

33. A non-pressurised pharmaceutical liquid solution spray formulation comprising:
(i) buprenorphine;
(ii) a solvent comprising ethanol; and
(iii) one or more antioxidants each of a molar ratio of antioxidant:buprenorphine between 0.2:1 and 25:1.

34. A formulation according to claim 33 wherein buprenorphine is employed in the form of its free base.

35. A formulation according to claim 34 wherein the concentration of buprenorphine is between 0.1-4% w/v.

36. A formulation according to claim 33 wherein the one or more antioxidants are selected from alkyl gallates, butylated hydroxyanisole, butylated hydroxytoluene, nordihydroguaiaretic acid, alpha-tocopherol, ascorbic acid and sodium metabisulfite.

37. A formulation according to claim 33 wherein the one or more antioxidants are selected from butylated hydroxyanisole, butylated hydroxytoluene, alpha-tocopherol, and ascorbic acid.

38. A formulation according to claim 33 which comprises only one antioxidant.

39. A formulation according claim 38 wherein the one antioxidant is butylated hydroxytoluene.

40. A formulation according to claim 33 which comprises two antioxidants.

41. A formulation according to claim 40 which comprises ascorbic acid and alpha-tocopherol.

42. A formulation according to claim 33 wherein the molar ratio of total antioxidant:buprenorphine between 0.2:1 and 25:1.

43. A formulation according to claim 33 characterised in that the formulation is substantially free of chloride.

44. A formulation according to claim 33 wherein the concentration of ethanol in the solvent is greater than 90% w/w.

45. A formulation according to claim 33 which is substantially free of water.

46. A formulation according to claim 44 wherein the concentration of ethanol in the solvent is around 100% w/w.

47. A formulation according to claim 33 wherein the concentration of ethanol in the solvent is approximately 30-90% w/w, the balance being water.

48. A formulation according to claim 33 wherein the pH of the formulation is between around 4 and 9.5.

49. A formulation according to claim 48 wherein the pH of the formulation is between around 5 and 7.

50. A formulation according to claim 48 wherein the pH of the formulation is between around 7 and 9.

51. A formulation according to claim 33 which contains saccharin.

52. A formulation according to claim 33 which contains saccharin sodium.

53. A formulation according to claim 33 which contains menthol.

54. A formulation according to claim 33 which contains peppermint oil.

55. A formulation according to claim 33 for transmucosal administration as a spray.

56. A formulation according to claim 55 wherein the transmucosal administration is sublingual administration.

57. A method for the treatment or prevention of opiate dependency or abuse or pain, the method comprising administering to a patient in need of such treatment an effective amount of a formulation according to claim 33.

58. A sealed container containing a plurality of doses of a formulation according to claim 33.

59. A container according to claim 58 which is made out of glass.

60. A metered dose dispensing system comprising a sealed container according to claim 58 fitted with a metering pump, an actuator and a channeling device.

61. A metered dose dispensing system according to claim 60 containing a metering chamber which is adapted for dispensation with the container in the upright orientation and wherein the metering chamber is in communication with the formulation by means of a dip-tube.

62. A metered dose dispensing system according to claim 60 adapted for transmucosal administration of the formulation as a spray.

63. A metered dose dispensing system according to claim 62 wherein the transmucosal administration is sublingual administration.

64. A process for preparation of formulations of the invention which comprises:
(a) taking buprenorphine in the form of its base and antioxidant and a solvent comprising ethanol optionally containing the other formulation ingredients and dissolving the buprenorphine and antioxidant in the solvent; or
(b) taking buprenorphine in the form of its base and antioxidant and a solvent comprising ethanol and dissolving the buprenorphine and antioxidant in the solvent, then adding the other formulation; or
(c) taking buprenorphine in the form of its base and a solvent comprising ethanol containing antioxidant and optionally containing the other formulation ingredients and dissolving the buprenorphine in the solvent; or
(d) taking buprenorphine in the form of its base and a solvent comprising ethanol and dissolving the buprenorphine in the solvent, then adding the other formulation ingredients; or
(e) the process of (a), (b), (c) or (d) in which the pH of the solvent is adjusted once all the other formulation ingredients are mixed together.
